SEQUENCE LISTING (2 sequences)

SEQ ID NO: 1 - protein, 8 aa, Artificial
  Synthetic peptide
TEEISEVK

SEQ ID NO: 2 - protein, 6 aa, Artificial
  Synthetic peptide
MDAEFR

(19) United States
(12) Patent Application Publication
(10) Pub. No.: US 2010/0021938 A1
(43) Pub. Date: Jan. 28, 2010

(54) Diagnosis and Prediction of Alzheimer's Disease

(76) Inventor: Olivier Boutaud, Nashville, TN (US)

Correspondence Address: FULBRIGHT & JAWORSKI L.L.P., 600 CONGRESS AVE., SUITE 2400, AUSTIN, TX, 78701, US

(21) Appl. No.: 12/502,476

(22) Filed: Jul. 14, 2009

Related U.S. Application Data
(60) Provisional application No. 61/083,686, filed on Jul. 25, 2008.

(51) Int. Cl. G01N 33/566; G01N 33/00; G01N 33/53

(52) U.S. Cl. 435/7.2; 436/86; 435/7.92

(57) ABSTRACT

Alzheimer's disease (AD) is characterized by the accumulation of amyloid-β peptide (Aβ) in the brain. Aβ is derived from amyloid precursor protein (APP) by β- and γ-secretases, with the β form (sAPPβ) being associated with the disease state, and the α form (sAPPα) being associated with the non-disease state. The present inventor proposes that defined the ratio of sAPPα to sAPPβ or the ratio of CTFα to CTFβ provide and accurate diagnosis of the disease, as well as a predictor for asymptomatic patients at risk of developing AD. In addition, drug screening and monitoring of treatment effectiveness can exploit this same sAPPα/sAPPβ or CTFα/CTFβ ratio.

This application claims benefit of priority to U.S. Provisional Application Ser. No. 61/083,686, filed Jul. 25, 2008, the entire contents of which are hereby incorporated by reference.

### BACKGROUND OF THE INVENTION

I. Field of the Invention

The present invention relates generally to the fields of neuropathology and molecular biology. More particularly, it concerns the use of sAPPα/sAPPβ and αCTF/βCFT ratios to diagnose and predict Alzheimer's Disease.

II. Description of Related Art

Neurodegenerative diseases are generally characterized by the loss of neurons from one or more regions of the central nervous system. One type of neurodegenerative disease is Alzheimer's disease (AD), the most common form of dementia among older people. Scientists believe that up to 4 million Americans suffer from AD. The disease usually begins after age 60, and risk goes up with age. While younger people also may get AD, it is much less common. About 3 percent of men and women ages 65 to 74 have AD, and nearly half of those age 85 and older may have the disease. While the subject of intensive research, the precise causes of AD are still unknown, it is challenging to diagnose, and there is no cure.

AD attacks parts of the brain that control thought, memory, and language. It was named after Dr. Alois Alzheimer, a German doctor. In 1906, Dr. Alzheimer noticed changes in the brain tissue of a woman who had died of an unusual mental illness. He found abnormal clumps, now called amyloid “plaques,” and tangled bundles of fibers, now called neurofibrillary “tangles.” Today, these plaques and tangles in the brain are considered hallmarks of AD.

The production, aggregation, and accumulation of amyloid β-protein (Aβ), the major constituent of the amyloid plaque, in the brain are initial steps in the pathogenesis of AD. Aβ is generated by the intracellular processing of amyloid β precursor protein (APP; FIG. 1) (Selkoe, 2001), a type I membrane protein (Kang et al., 1987), by proteases β-secretase (memapsin 2 or BACE1) and γ-secretase. The cytoplasmic domain of APP (APPcyt), through its interactions with cytoplasmic proteins, plays an important role in the regulation of APP metabolism and Aβ production (King and Turner, 2004).

### SUMMARY OF THE INVENTION

In accordance with the present invention, there is provided a method for assessing the risk of development or diagnosing the presence of Alzheimer's Disease (AD) in a subject comprising (a) obtaining a sample from the subject that contains soluble APP (sAPP) or that contains C-terminal fragment of APP (CTF); and (b) assessing sAPPα to sAPPβ ratio or αCTF to βCTF ratio in the sample, wherein a decreased sAPPα to sAPPβ ratio or a decreased αCTF to βCTF ratio is indicative of a decreased α-secretase activity with respect to β-secretase activity, which is indicative of risk or presence of AD. The sample may be cerebrospinal fluid, whole blood, plasma, serum or a biopsy. Assessing may comprise mass spectrometry, radioactive immunoassay (RIA), enzymatic immunoassay (EIA), enzyme-linked immunosorbent assay (ELISA), or Western blott analysis. The subject may or may not be exhibiting symptoms of AD. The method may further comprise subjecting the subject to standard AD diagnostic procedures.

The method may further comprising isolating sAPP or CTF prior to step (b). The method may further comprising digesting sAPP with a protease that generates at least one a distinct peptide from sAPPα or sAPPβ, such as MDAEFR. The protease may further produce at least one common peptide from sAPPα and sAPPβ, such as TEEISEVK. The protease may be trypsin or endoproteinase ArgC. Assessing may also comprises liquid chromatography and ESI mass spectrometry. The method may further comprise use of a labeled standard peptide, or an unlabeled standard peptide.

In another embodiment, there is provided a method for assessing efficacy of an Alzheimer's Disease (AD) treatment in a subject comprising (a) obtaining a first sample from the subject that contains soluble APP (sAPP) or that contains C-terminal fragment of APP (CTF); (b) obtaining a second sample from the subject that contains sAPP or CTF; and (c) assessing sAPPα to sAPPβ ratio or αCTF to βCTF ratio in both of the samples, wherein an increased sAPPα to sAPPβ ratio or αCTF to βCTF ratio in the second sample as compared to the first sample is indicative of a treatment efficacy. The sample may be cerebrospinal fluid, whole blood, plasma, serum or a biopsy. Assessing may comprise mass spectrometry, radioactive immunoassay (RIA), enzymatic immunoassay (EIA), enzyme-linked immunosorbent assay (ELISA), or Western blott analysis. The method may further comprise subjecting the subject to standard AD assessment procedures.

The method may further comprise isolating sAPP or CTF prior to step (b). The method may also further comprise digesting sAPP with a protease that generates at least one a distinct peptide from sAPPα or sAPPβ, such as MDAEFR. The protease may further produce at least one common peptide from sAPPα and sAPPβ, such as TEEISEVK. The protease may be trypsin or endoproteinase ArgC. Assessing comprises may also comprising liquid chromatography and ESI mass spectrometry. The method may further comprise the use of a labeled or unlabeled standard peptide. The first sample may be a pre-treatment sample and the second sample may be a post-treatment sample. Alternatively, the first and second samples may be post-treatment samples, but an additional treatment occurs following obtaining of the first sample but prior to obtaining the second sample. The first and second samples may also be post-treatment samples, but at different times after treatment to follow the progression of the therapeutic change.

In yet another embodiment, there is provided a method of screening a candidate substance for activity against Alzheimer's Disease (AD) comprising (a) providing a cell that produces soluble APP (sAPP) or C-terminal fragment of APP (CTF); (b) contacting the cell with the candidate substance; (c) obtaining sAPP or CTF from the cell; and (d) assessing sAPPα to sAPPβ ratio or αCTF to βCTF ratio in the sample, wherein an increase in sAPPα to sAPPβ ratio or αCTF to βCTF ratio is indicative of candidate substance activity against AD. The cell may produce a reduced sAPPα to sAPPβ ratio or αCTF to βCTF ratio as compared to a normal or non-diseased cell. Alternatively, the cells may be treated with a chemical to produce a reduced sAPPα to sAPPβratio or αCTF to βCTF ratio as compared to normal or non-diseased cells. The cell may be located in an animal subject, and contacting comprises administering the candidate substance to the animal. The animal subject may be further subjected to behavioral assessment. The cell may be a neuron, platelets, microglia or astrocyte.

In yet another embodiment, there is provided a method for assessing progression of Alzheimer's Disease (AD) in a subject comprising (a) obtaining a first sample from the subject that contains soluble APP (sAPP) or C-terminal fragment of APP (CTF); (b) obtaining a second sample from the subject that contains sAPP or CTF; and (c) assessing sAPPα to sAPPβ ratio or αCTF to βCTF ratio in both of the samples, wherein a decreased sAPPα to sAPPβratio or αCTF to βCTF ratio in the second sample as compared to the first sample is indicative of a AD progression.

In still yet another embodiment, there is provided a method for staging Alzheimer's Disease (AD) in a subject comprising (a) obtaining a sample from the subject that contains soluble APP (sAPP) or C-terminal fragment of APP (CTF); (b) assessing sAPPα to sAPPβratio or αCTF to βCTF ratio in the sample; and (c) comparing the sAPPα to sAPPβratio or αCTF to βCTF ratio to known ratios for stages of AD.

In a further embodiment, there is provided a method of treating a subject with Alzheimer's Disease comprising administering to the subject a prostanoid receptor agonist. The prostanoid receptor may be a thromboxane receptor or an isoform of a prostaglandin E₂ receptor. The agonist may be a partial agonist or allosteric modulator of the thromboxane receptor or the isoform of the prostaglandin E₂ receptor. In still a further embodiment, there is provided a method of treating a subject with Alzheimer's Disease comprising administering to the subject a prostanoid receptor antagonist. The antagonist may be an antagonist of a prostaglandin I₂ (or prostacyclin) receptor. The antagonist may be an allosteric modulator of the prostaglandin I₂ (or prostacyclin) receptor.

In even a further embodiment, there is provided a method of screening a prostanoid receptor agonist or antagonist for activity against Aβ production comprising (a) providing a cell that produces sAPP; (b) contacting the cell with a prostanoid receptor agonist or antagonist; and (c) assessing sAPPα to sAPPβ ratio or αCTF to βCTF ratio in the cell or cell culture thereof.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions and kits of the invention can be used to achieve methods of the invention.

Throughout this application, the term “about” is used to indicate that a value includes the inherent variation of error for the device, the method being employed to determine the value, or the variation that exists among the study subjects.

### BRIEF DESCRIPTION OF THE DRAWINGS

The following drawings form part of the present specification and are included to further demonstrate certain aspects of the present invention.

FIG. 1: Proteolytic processing of amyloid precursor protein (APP). APP is cleaved by either α-secretase (blue pathway) or by β-secretase (red pathway). The products of the first proteolysis are further processed by g-secretase to form p3 (non-toxic peptide, blue) or Ab (neurotoxic peptides, red), respectively. The secreted APP fragments (sAPPα and sAPPβ) are representative of the two secretase activities (α- and β-, respectively).

FIG. 2: sAPPα release is mediated through G protein-coupled receptors. Human platelets (600,000 cells/μl) were incubated with 20 μM arachidonic acid (AA), U46,619 (500 nM), PAR1-AP (100 μM), PAR4-AP (200 μM) or thrombin (1 U/ml) for 1 h. The cells were then pelleted at 10,000×g for 15 min and the levels of sAPPα in the supernatant were determined by Western blot analysis using the antibody 6E10 (Calbiochem).

FIG. 3: The effect of arachidonic acid is mediated by the TP receptor. Left panel: Human platelets were incubated by increasing concentrations of U46,619 and sAPPa was analyzed in the supernatant as described for FIG. 2. Right panel: Human platelets were pre-incubated for 15 min with increasing concentration of an antagonist of the TP receptor, SQ29,548, prior addition of 7.5 μM of arachidonic acid. sAPPα release was analyzed by western blot.

FIG. 4: Activation of PGI₂ receptor inhibits sAPPa release triggered by U46,619. Human platelets were pre-incubated for 30 min with increasing concentrations of carbaprostacylin prior addition of 500 nM of U46,619. After 1 h the levels of sAPPa in the supernatant were determined by western blot analysis. Protein bands were scanned and the pixels integrated. sAPPa release is expressed as a percent of maximum where no CP was added.

FIG. 5: PGE₂ potentiates the effect of a sub-efficacious concentration of arachidonic acid. Washed platelets were pre-incubated for 2 min with increasing concentrations of PGE₂ or vehicle prior addition of 0.5 μM of arachidonic acid. After 1 h, sAPPα release in the supernatant was determined by Western blot analysis as described in FIG. 2. Protein bands were scanned and the pixels integrated. sAPPα release is expressed as a percent of the maximum obtained with 7.5 nM PGE₂ and 0.5 μM arachidonic acid.

FIG. 6: Chromatograms of the two prominent ion products of tryptic peptides from sAPPs. After digestion with trypsin, sAPPa and sAPPb were analyzed by LC/ESI/MS/MS as indicated above. Chromatograms for the product ions b2 and y6 for the peptide TEEISEVK and y4 and y5 for the peptide MDAEFR are shown.

FIG. 7: Amino acid sequence of APP at the Ab site. The cleavage sites for the three secretases are indicated by an arrow. The two peptides identified by proteomics analysis are indicated (TEEISEVK (SEQ ID NO:1) and MDAEFR (SEQ ID NO:2)). The sequence of Aβ₁₋₄₂ is underlined.

FIG. 8: Specificity of the two peptides for the two sAPP isoforms. Peptides obtained by digestion of a total of 100 ng of sAPPs were analyzed as described above. The product ions y6 and y4 were selected for TEEISEVK and MDAEFR, respectively. Chromatograms of these two product ions are shown for each sAPP isoform.

FIG. 9: Correlation between the ratios of the different product ions and the ratios of the different sAPPs. Ratios of the abundances of the different product ions are represented as a function of the ratios of the two sAPPs mixed in the samples.

FIG. 10: Detection of the two sAPP isoforms released from human platelets. 300 μl of human washed platelets at 600,000 cells/μl were activated with 500 nM U46,619. sAPPs released in the medium were immunoprecipitated and digested with trypsin. The peptides were analyzed as described above. The product ions y6 and y4 were monitored for TEEISEVK and MDAEFR, respectively. Chromatograms of the two product ions are shown for each sAPP isoform.

FIG. 11: Detection of the two sAPP isoforms present in human CSF. Human CSF from a patient with AD (AD) and from an age-matched control patient (control) were diluted with three volumes of PBS and the sAPPs present in the biological fluids were immunoprecipitated and digested with 1 Unit of modified trypsin. 150 pg of MDAE[¹³C]-FR and TEE[¹³C]-ISEVK were added as internal standards and the samples were analyzed by LC/ESI/MS/MS using the LCQ Deca XP ion trap mass spectrometer as described above. The product ions y6 and y4 were monitored for TEEISEVK and MDAEFR, respectively. Chromatograms of these two product ions are shown for each sample.

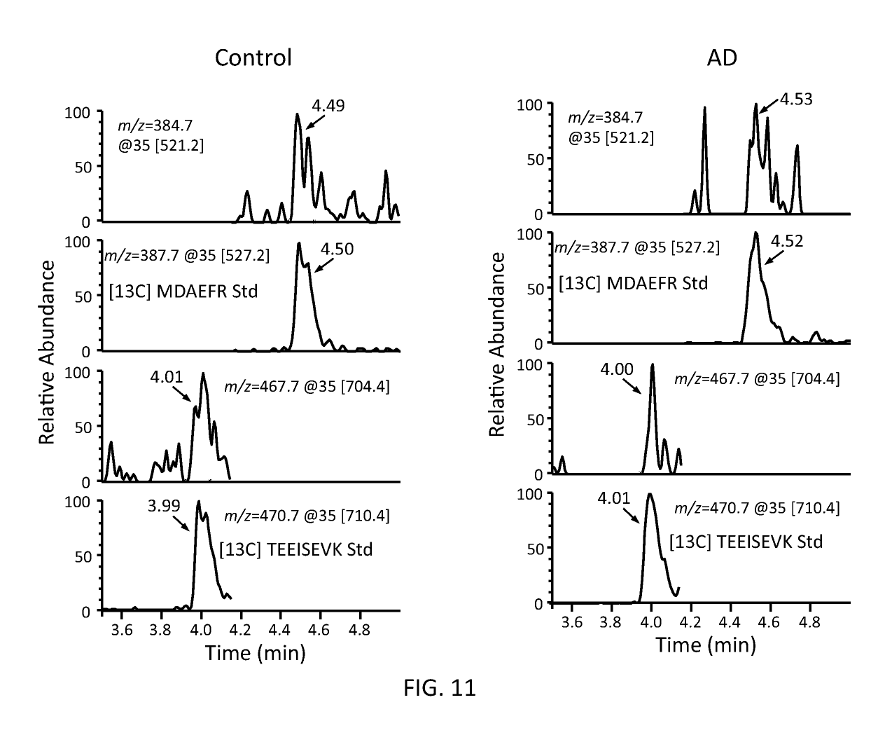

FIG. 12: The ratio sAPPα/sAPPβ from human CSF correlates with Braak stage. Human CSF from patient s with different Braak stages were diluted with three volumes of PBS and the sAPPs present in the biological fluids were immunoprecipitated and digested with 1 Unit of modified trypsin. After analysis of the samples by LC/ESI/MS/MS using the LCQ Deca XP ion trap mass spectrometer as below for FIG. 11, the product ions y6 and y4 were monitored for TEEISEVK and MDAEFR, respectively. The ratio sAPPα/sAPPβ were calculated and analyzed versus the severity of the disease as indicated by the Braak stage.

FIG. 13: Linearity of the assay of sAPP's peptides by LC/MS/MS. Synthetic peptides (TEEISEVK and MDAEFR) were weighed and diluted in ambic containing 1% lysine. Stable isotopic peptides (100 pg) were added as internal standards and the two peptides were quantified by LC/MS/MS. Each bar is the means±S.E.M. for 4 independent measurements. Dotted lines indicate the 95% confidence intervals for each standard curve.

FIG. 14: Reproducibility of the LC/MS/MS method using human CSF. Proteins in human CSF (50 μl) were digested with 2.5 U of trypsin for two times 24 hours. Stable isotopic peptides were added to the samples as internal standards and the two peptides (TEEISEVK and MDAEFR) were quantified by LC/MS/MS. Each bar is the means±S.E.M. for 3 independent measurements

FIGS. 15A-B: Validation of the LC/MS/MS method using platelets and Western blot analysis of secreted APPs. Alpha-secretase activity was activated with an agonist of the thromboxane receptor (U46) in platelets. At different times, the platelets were centrifuged and the supernatant was used to measure the release of secreted-APPa by Western blot analysis (FIG. 15A) and by measuring secreted-APPa by LC/MS/MS (FIG. 15B). sAPPa expressed as % of the maximum release at 40 min.

FIG. 16: Reproducibility of the LC/MS/MS method using human platelets. Human washed platelets (600,000 cells/μl) were activated with U46,619. At the different times, the sAPPs secreted by the platelets were digested with 2.5 U of trypsin for two times 24 hours. Stable isotopic peptides were added to the samples as internal standards, the two peptides (TEEISEVK and MDAEFR) were quantified by LC/MS/MS and the amount of sAPPs were calculated. Each value is the means±S.E.M. for 3 independent measurements.

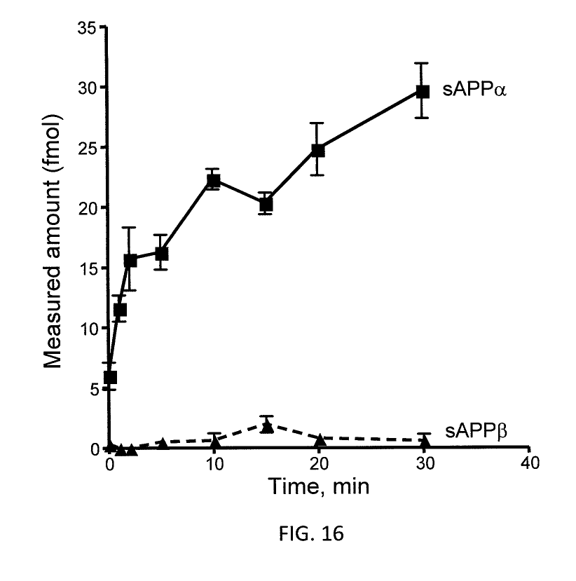

### DESCRIPTION OF ILLUSTRATIVE EMBODIMENTS

### I. The Present Invention

Much research progress has been made in understanding the mechanisms and molecules implicated in Alzheimer's disease (AD), but there are many unmet needs. First, reliable biomarkers of the disease that permit early diagnosis prior to cognitive decline are still lacking (Fagan et al., 2005; Galasko, 2005). Second, clinical trials examining new therapies rely on clinical rating scales that measure cognitive performance. These are subject to great variability that makes it difficult to discriminate between symptomatic and biological effects. Third, an efficacious treatment must be initiated before the loss of brain tissue has occurred, time at which the diagnosis of AD often represents a challenge for clinicians. Indeed, at early stages of AD, deficits are mild and often overlap with changes associated with aging, general medical disorders or other types of dementia. Taken together, these arguments indicate the need for development of biomarkers that can be used reliably at early stages of AD.

In 1998, a Working Group convened by the Alzheimer's Association and the National Institute on Aging proposed a set of guidelines for biomarkers for AD (Consensus Report of the Working Group, 1998). The key elements of their recommendations were that biomarkers should reflect a basic pathogenic or pathologic feature of Alzheimer's Disease; needed to be reliable; should have sensitivity and specificity of at least 80%; should be present even in very mild AD; and the diagnostic accuracy should be verified in autopsy-confirmed patients. While the ideal has not been met, certain markers currently available satisfy some of these proposed criteria.

The measurement of sAPPα alone indicates that the mean levels are significantly lower in AD, but there is significant overlap with control values, preventing its use as a prognostic tool (Colciaghi et al., 2002; Tang et al., 2006). Colciaghi et al. presented the most compelling evidence for measuring sAPPα as a biomarker for AD (Colciaghi et al., 2002) by studying a small subset of patients with probable AD, subset which is not representative of the general population afflicted with the disease. However, even with this very restricted population, they had substantial overlap between AD and control values. Taken together, these results indicate that when applied more generally to all patients with AD, it is unlikely that the measurement of sAPPα alone would provide the statistical significance necessary for its use as a biomarker.

The inventor hypothesized that the abundance of sAPPα relative to sAPPβ could constitute a reliable biomarker for AD. Accordingly, he developed a method using liquid chromatography coupled to mass spectrometry that allows the measurement of relative amounts of sAPPα to sAPPβ from complex protein mixtures with high sensitivity. In a specific embodiment, secreted APPs are immunoprecipitated and digested with trypsin, which generates peptides specific for the two secreted species of APP. This method presents the following advantages. First, the two markers are measured simultaneously and the use of isotopically labeled internal standards allows accurate quantitative measurements of the two peptides within the same sample. Second, digestion with trypsin yields peptides that are specific for each species and the generation of fragments depending on the amino acid sequence of the peptide permits unequivocal characterization of the measured peptides. Third, sAPPα and sAPPβ are soluble proteins that can be easily isolated from body fluids and analyzed by mass spectrometry. And fourth, LC/ESI/MS/MS is a highly accurate technique that allows us to measure as little as 1 pmol of the two peptides.

The development off AD is a chronic process that starts many years before the appearance of symptoms. There are no markers for risk factors for the development of sporadic AD. Measurement of the ratio of sAPPs in patients in their late 40s to early 60s provides information about the risk of developing the disease later in life. Thus, the present invention can be used for the prediction of developing AD, as well as it's early diagnosis. These and other aspects of the invention are described below.

### II. Alzheimer's Disease

A. Background

AD is a progressive, neurodegenerative disease characterized by memory loss, language deterioration, impaired visuospatial skills, poor judgment, indifferent attitude, but preserved motor function. AD usually begins after age 65, however, its onset may occur as early as age 40, appearing first as memory decline and, over several years, destroying cognition, personality, and ability to function. Confusion and restlessness may also occur. The type, severity, sequence, and progression of mental changes vary widely. The early symptoms of AD, which include forgetfulness and loss of concentration, can be missed easily because they resemble natural signs of aging. Similar symptoms can also result from fatigue, grief, depression, illness, vision or hearing loss, the use of alcohol or certain medications, or simply the burden of too many details to remember at once.

There is no cure for AD and only limited ability to slow the progression of the disease. For some people in the early or middle stages of the disease, medication such as tacrine may alleviate some cognitive symptoms. Aricept (donepezil) and Exelon (rivastigmine) are reversible acetylcholinesterase inhibitors that are indicated for the treatment of mild to moderate dementia of the Alzheimer's type. Also, some medications may help control behavioral symptoms such as sleeplessness, agitation, wandering, anxiety, and depression. These treatments are aimed at making the patient more comfortable. AD is a progressive disease, and the course of the disease varies from person to person. Some people have the disease only for the last 5 years of life, while others may have it for as many as 20 years. The most common cause of death in AD patients is infection.

The molecular aspect of AD is complicated and not yet fully defined. As stated above, AD is characterized by the formation of amyloid plaques and neurofibrillary tangles in the brain, particularly in the hippocampus which is the center for memory processing. Several molecules contribute to these structures: amyloid β protein (Aβ), presenilin (PS), cholesterol, apolipoprotein E (ApoE), and Tau protein. Of these, Aβ appears to play the central role.

Aβ contains approximately 40 amino acid residues. The 42 and 43 residue forms are much more toxic than the 40 residue form. Aβ is generated from an amyloid precursor protein (APP) by sequential proteolysis. One of the enzymes lacks sequence specificity and thus can generate Aβ of varying (39-43) lengths. The toxic forms of Aβ cause abnormal events such as apoptosis, free radical formation, aggregation and inflammation. Presenilin encodes the protease responsible for cleaving APP into Aβ. There are two forms—PS1 and PS2. Mutations in PS1, causing production of Aβ₄₂, are the typical cause of early onset AD.

Cholesterol-reducing agents have been alleged to have AD-preventative capabilities, although no definitive evidence has linked elevated cholesterol to increased risk of AD. However, the discovery that Aβ contains a sphingolipid binding domain lends further credence to this theory. Similarly, ApoE, which is involved in the redistribution of cholesterol, is now believed to contribute to AD development. As discussed above, individuals having the ApoE4 allele, which exhibits the least degree of cholesterol efflux from neurons, are more likely to develop AD.

Tau protein, associated with microtubules in normal brain, forms paired helical filaments (PHFs) in AD-affected brains which are the primary constituent of neurofibrillary tangles. Recent evidence suggests that Aβ proteins may cause hyperphosphorylation of Tau proteins, leading to disassociation from microtubules and aggregation into PHFs.

B. APP

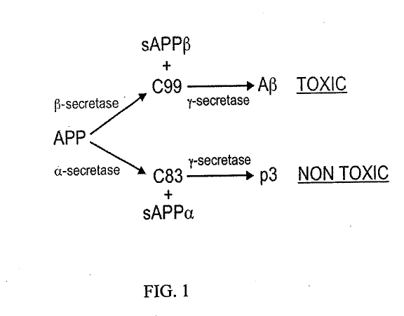

The amyloid precursor protein (APP) is a ubiquitous type 1 transmembrane protein abundantly expressed in neurons and in platelets. It is involved in neural development, in hemostasis, and possibly in the migration and differentiation of macrophages into foam cells. APP is processed by a series of proteases that are part of two parallel and exclusive pathways. The pathways are determined by the first proteolytic cleavage (FIG. 1). If APP is first cleaved by the α-secretase, it generates a neurotrophic and neuroprotective fragment, sAPPα. Further cleavage by the γ-secretase leads to the formation of a non toxic peptide, p3. If the first cleavage is made by the β-secretase, it generates a fragment called sAPPβ and further processing of APP by the γ-secretase leads to the generation of a series of toxic peptides, Aβ1-xx.

In the brain, sAPPα promotes neuronal survival, adhesive interactions, neurite outgrowth, synaptogenesis, and synaptic plasticity (for review: De Strooper and Annaert, 2000). Importantly, if α-secretase cleaves APP, it precludes cleavage by the β-secretase, preventing formation of the amyloid β species. Activation by G-protein-coupled receptors (GPCRs) has been shown to activate sAPPα release by neurons (Nitsch et al., 2000; Nitsch et al., 1992), and by platelets (Skovronsky et al., 2001). However the signaling mechanism underlying this regulation is still not understood. sAPPα is also present in large amounts in platelets where it is present in the α-granules and is released upon stimulation of the cells.

C. CTF

APP is processed by a series of proteases that are part of two parallel and exclusive pathways. The pathways are determined by the first proteolytic cleavage (FIG. 1). If APP is first cleaved by the α-secretase, it generates two fragments: a soluble neurotrophic and neuroprotective fragment, sAPPα, and a membrane bound C-terminal fragment (α-CTF). Further cleavage by the γ-secretase leads to the formation of a non toxic peptide, p3. If the first cleavage is made by the β-secretase, it generates two equivalent peptides called sAPPβ and β-CTF, and further processing of β-CTF by the γ-secretase leads to the generation of a series of toxic peptides, Aβ1-xx. According to this mechanism, the relative activities of α- to β-secretases can be monitored by measuring not only the ratio of sAPPα to sAPPβ, but as well as the ratio of α-CTF to β-CTF. To determine variations in α-secretase activity, one can measure sAPPα/sAPPβ ratio in the supernatant, or α- and β-CTFs in the pelleted cells.

D. α-Secretase

The proteolytic cleavage yielding sAPPα is catalyzed by the α-secretase. Several members of the family of membrane-bound disintegrin metalloproteinases (ADAMs) are known to harbor α-secretase activity. This activity consists of a constitutive component and a regulated component that can be activated by protein kinase C (PKC) (Buxbaum et al., 1998). Three members of the ADAMs have been shown to be up regulated by activation of PKC: the TNF-α converting enzyme (TACE/ADAM17), ADAM10, and MDC9. Among them, ADAM10 is thought to be responsible for the majority of the α-secretase activity. Several lines of evidence indicate that α-secretase is a potential therapeutic target for AD (Hooper and Turner, 2002). Moderate expression of ADAM10 in transgenic APP (V7171) mice almost completely prevents formation of amyloid plaques (Postina et al., 2004). In cells, cleavage of APP by α-secretase is under control of several major neurotransmitter and neurotrophic molecules (Hooper and Turner, 2002; Kojro and Fahrenholz, 2005; Kojro et al., 2006). The selective M₁ muscarinic receptor (M₁mAChR)-agonist AF102B has been shown to decrease the levels of amyloid in cerebrospinal fluid from patients with AD (Nitsch et al., 2000) and its analog AF267B decreases APP processing via the β-secretase pathway and decreases development of amyloid pathology in 3xTg-AD mice (Caccamo et al., 2006). Interestingly, it also increases expression of ADAM 17/TACE (Caccamo et al., 2006).

The decrease of α-secretase activity has been reported to occur at early stages of AD (Colciaghi et al., 2002; Tang et al., 2006). In cells, increased non-amyloidogenic α-secretase activity results in decreased secretion of Aβ (Kojro et al., 2001). sAPPα levels decrease in AD whereas sAPPβ levels increase (Tang et al., 2006). Understanding which molecular mechanisms responsible for α-secretase activation are failing during the development of AD may lead to the discovery of new therapeutic targets for drugs aiming at restoring normal α-secretase activity in the brain. Activation of α-secretase activity in vivo may represent a safer alternative to inhibiting β- or γ-secretase, which could lead to accumulation of APP in the membranes or to the inhibition of other physiological pathways in which these two proteases could be involved.

There are 16 different G proteins α-subunits divided in 4 families: Gs stimulates adenylate cyclase; G_{i/o} inhibits adenylate cyclase; G_{q/11} stimulates phospholipase Cβ; and G_{12/13} stimulates Rho-guanine nucleotide exchange factors. As with many other G protein coupled receptors, prostanoids receptors couple to multiple G proteins. The thromboxane (TP) receptor is known to couple to G_{q} (Shenker et al., 1991), G₁₁ (Kinsella et al., 1997), and G_{12/13} (Djellas et al., 1999; Offermanns et al., 1994). The prostacyclin (IP) receptor has been shown to couple to Gₛ (Adie et al., 1992) or to Gq (Schwaner et al., 1995) depending on the cell types used. In platelets, prostacyclin inhibits thromboxane-induced aggregation via stimulation of adenylate cyclase through Gₛ. PGE₂ mediates its various effects through 4 receptor sub-types. The EP 1 receptor likely couples to G_{q} (Hebert et al., 1990). Although both EP2 and EP4 seem to couple to Gₛ (Honda et al., 1993; Regan et al., 1994), they have different pharmacological binding capacities and different signal transduction pathways. Finally, EP3 includes several splice variants differing in their C-terminal cytoplasmic tails. Although it was first observed that EP3 couples to Gᵢ (Sonnenburg et al., 1990), some data indicate that the receptors also signal through the small G-protein Rho (Katoh et al., 1998).

Interestingly, despite the vast amount of research on the different prostanoid receptors, the understanding of their signal transduction pathways in the brain remains unclear. Recognition of the roles of prostaglandin receptors in brain physiology and pathophysiology is starting to emerge for PGE₂ (Kehner et al., 2008; Liang et al., 2005) and thromboxane (TP, (Nishihara et al., 2000; Ramamurthy et al., 2006; Shineman et al., 2008)), but little is know about the role of the prostacyclin receptor. The inventor has obtained preliminary data in platelets indicating that APP processing is strongly regulated by the interplay of different prostanoid receptors. Activation of the TP receptor is a potent activator of sAPPα release; this effect is completely inhibited by a specific antagonist of the TP receptor. Because TP receptor and M₁mAChR have similar signal transduction pathways, we propose to investigate whether TP receptor activation modifies APP processing in PC12 cells in culture and in mouse hippocampus. These results in platelets also show that activation of the IP receptor prevents the activation of α-secretase triggered by the TP receptor activation. IP receptor activation leads to activation of the protein kinase A via increase of intracellular cAMP levels. Accordingly, the inventor has hypothesized that activation of the Gs-coupled IP or EP2/EP4 receptors in the brain may decrease the activation of α-secretase triggered by M₁mAChR agonists. Finally, activation of the Gᵢ-coupled EP3 receptor in platelets potentiates the effect of the TP agonist U46,619. By analogy to the platelets, EP3 activation in brain could potentiate the effect of M₁ mAChR by inhibiting intracellular cAMP production.

### III. Methods for Assessing sAPP or CTF Ratios

The present invention involves, in one aspect, the assessment of sAPP or CTF ratios in samples from subjects that may be suffering from or at risk of developing Alzheimer's Disease (AD). In other embodiments, one may use these methods to assessing the efficacy of an AD treatment, or to screen agents for the ability to modulate these ratios, and hence the production of Aβ and AD plaques resulting therefrom.

Samples to be assessing include cerebrospinal fluid and brain tissue (from biopsy, cadavers or experimental animals). sAPP and CTF are produced by platelets upon activation of the cells, and thus whole blood, plasma, and serum are also suitable sample sources.

A. Protein Purification

It may be desirable to purify proteins from a sample as part of the present invention. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity), both before and after other procedures (e.g., proteolysis). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention may concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

A variety of techniques suitable for use in protein purification will be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (e.g., alter pH, ionic strength, and temperature).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand also should provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

B. Protease Treatment

The present invention involves, in one aspect, the proteolytic breakdown of target proteins into unique peptides for further analysis, e.g., by mass spectrometry. The use of appropriate proteases will produce such unique peptides which can then be identified and quantitated in follow-on procedures. Such proteases include trypsin and endoproteinase Arg C. This treatment will, in most cases, follow a first round of purification in which proteins are separated from non-proteins in a patient sample.

Trypsin is a serine protease found in the digestive system, where it breaks down proteins. Trypsin predominantly cleaves peptide chains at the carboxyl side of the amino acids lysine and arginine, except when either is followed by proline. It is produced in the pancreas in the form of inactive zymogen, trypsinogen, and then secreted into the small intestine, where the enzyme enteropeptidase activates it into trypsin by proteolytic cleavage. The resulting trypsins themselves activate more trypsinogens (autocatalysis), so only a small amount of enteropeptidase is necessary to start the reaction. This activation mechanism is common for most serine proteases, and serves to prevent autodigestion of the pancreas.

Endoproteinase Arg C, also known as Clostripain, is a sulfhydryl proteinase associated with collagenase and isolated from Clostridium histolyticum. It is highly specific for the carboxyl peptide bond of arginine. Gilles et al. (1979) indicate it to be composed of two chains with relative molecular masses of 45,000 and 12,500. Studies on the active site are reported by Porter et al. (1971). Optimum pH is 7.4-7.8 (activity against a-benzoyl-arginine ethyl ester). Inhibitors include oxidizing agents and sulfhydryl reactants, as well as Co²⁺, Cu²⁺, Cd²⁺, and heavy metal ions. Citrate, borate, and Tris anions partially inhibit. TLCK reacts with active site. (Porter, et al., 1971). Sulfhydryl requirement are satisfied by dithiothreitol, cysteine, or other reducing agents. Calcium ion is essential.

C. Mass Spectrometry

By exploiting the intrinsic properties of mass and charge, mass spectrometry (MS) can resolved and confidently identified a wide variety of complex compounds, including proteins. Traditional quantitative MS has used electrospray ionization (ESI) followed by tandem MS (MS/MS) (Chen et al., 2001; Zhong et al., 2001) while newer quantitative methods are being developed using matrix assisted laser desorption/ionization (MALDI) followed by time of flight (TOF) MS (Bucknall et al., 2002; Mirgorodskaya et al., 2000; Gobom et al., 2000). In accordance with the present invention, one can generate mass spectrometry profiles for peptides that are unique for various forms of APP.

1. ESI

ESI is a convenient ionization technique developed by Fenn and colleagues (Fenn et al., 1989) that is used to produce gaseous ions from highly polar, mostly nonvolatile biomolecules, including lipids. The sample is injected as a liquid at low flow rates (1-10 μL/min) through a capillary tube to which a strong electric field is applied. The field generates additional charges to the liquid at the end of the capillary and produces a fine spray of highly charged droplets that are electrostatically attracted to the mass spectrometer inlet. The evaporation of the solvent from the surface of a droplet as it travels through the desolvation chamber increases its charge density substantially. When this increase exceeds the Rayleigh stability limit, ions are ejected and ready for MS analysis.

A typical conventional ESI source consists of a metal capillary of typically 0.1-0.3 mm in diameter, with a tip held approximately 0.5 to 5 cm (but more usually 1 to 3 cm) away from an electrically grounded circular interface having at its center the sampling orifice, such as described by Kabarle et al. (1993). A potential difference of between 1 to 5 kV (but more typically 2 to 3 kV) is applied to the capillary by power supply to generate a high electrostatic field (10⁶ to 10⁷ V/m) at the capillary tip. A sample liquid carrying the analyte to be analyzed by the mass spectrometer, is delivered to tip through an internal passage from a suitable source (such as from a chromatograph or directly from a sample solution via a liquid flow controller). By applying pressure to the sample in the capillary, the liquid leaves the capillary tip as a small highly electrically charged droplets and further undergoes desolvation and breakdown to form single or multicharged gas phase ions in the form of an ion beam. The ions are then collected by the grounded (or negatively charged) interface plate and led through an the orifice into an analyzer of the mass spectrometer. During this operation, the voltage applied to the capillary is held constant. Aspects of construction of ESI sources are described, for example, in U.S. Pat. Nos. 5,838,002; 5,788,166; 5,757,994; RE 35,413; and 5,986,258.

2. ESI/MS/MS

In ESI tandem mass spectroscopy (ESI/MS/MS), one is able to simultaneously analyze both precursor ions and product ions, thereby monitoring a single precursor product reaction and producing (through selective reaction monitoring (SRM)) a signal only when the desired precursor ion is present. When the internal standard is a stable isotope-labeled version of the analyte, this is known as quantification by the stable isotope dilution method. This approach has been used to accurately measure pharmaceuticals (Zweigenbaum et al., 2000; Zweigenbaum et al., 1999) and bioactive peptides (Desiderio et al., 1996; Lovelace et al., 1991). Newer methods are performed on widely available MALDI-TOF instruments, which can resolve a wider mass range and have been used to quantify metabolites, peptides, and proteins. Larger molecules such as peptides can be quantified using unlabeled homologous peptides as long as their chemistry is similar to the analyte peptide (Duncan et al., 1993; Bucknall et al., 2002). Protein quantification has been achieved by quantifying tryptic peptides (Mirgorodskaya et al., 2000). Complex mixtures such as crude extracts can be analyzed, but in some instances sample clean up is required (Nelson et al., 1994; Gobom et al., 2000).

3. SIMS

Secondary ion mass spectroscopy, or SIMS, is an analytical method that uses ionized particles emitted from a surface for mass spectroscopy at a sensitivity of detection of a few parts per billion. The sample surface is bombarded by primary energetic particles, such as electrons, ions (e.g., O, Cs), neutrals or even photons, forcing atomic and molecular particles to be ejected from the surface, a process called sputtering. Since some of these sputtered particles carry a charge, a mass spectrometer can be used to measure their mass and charge. Continued sputtering permits measuring of the exposed elements as material is removed. This in turn permits one to construct elemental depth profiles. Although the majority of secondary ionized particles are electrons, it is the secondary ions which are detected and analysis by the mass spectrometer in this method.

4. LD-MS and LDLPMS

Laser desorption mass spectroscopy (LD-MS) involves the use of a pulsed laser, which induces desorption of sample material from a sample site—effectively, this means vaporization of sample off of the sample substrate. This method is usually only used in conjunction with a mass spectrometer, and can be performed simultaneously with ionization if one uses the right laser radiation wavelength.

When coupled with Time-of-Flight (TOF) measurement, LD-MS is referred to as LDLPMS (Laser Desorption Laser Photoionization Mass Spectroscopy). The LDLPMS method of analysis gives instantaneous volatilization of the sample, and this form of sample fragmentation permits rapid analysis without any wet extraction chemistry. The LDLPMS instrumentation provides a profile of the species present while the retention time is low and the sample size is small. In LDLPMS, an impactor strip is loaded into a vacuum chamber. The pulsed laser is fired upon a certain spot of the sample site, and species present are desorbed and ionized by the laser radiation. This ionization also causes the molecules to break up into smaller fragment-ions. The positive or negative ions made are then accelerated into the flight tube, being detected at the end by a microchannel plate detector. Signal intensity, or peak height, is measured as a function of travel time. The applied voltage and charge of the particular ion determines the kinetic energy, and separation of fragments are due to different size causing different velocity. Each ion mass will thus have a different flight-time to the detector.

One can either form positive ions or negative ions for analysis. Positive ions are made from regular direct photoionization, but negative ion formation require a higher powered laser and a secondary process to gain electrons. Most of the molecules that come off the sample site are neutrals, and thus can attract electrons based on their electron affinity. The negative ion formation process is less efficient than forming just positive ions. The sample constituents will also affect the outlook of a negative ion spectra.

Other advantages with the LDLPMS method include the possibility of constructing the system to give a quiet baseline of the spectra because one can prevent coevolved neutrals from entering the flight tube by operating the instrument in a linear mode. Also, in environmental analysis, the salts in the air and as deposits will not interfere with the laser desorption and ionization. This instrumentation also is very sensitive, known to detect trace levels in natural samples without any prior extraction preparations.

5. MALDI-TOF-MS

Since its inception and commercial availability, the versatility of MALDI-TOF-MS has been demonstrated convincingly by its extensive use for qualitative analysis. For example, MALDI-TOF-MS has been employed for the characterization of synthetic polymers (Marie et al, 2000; Wu et al., 1998). peptide and protein analysis (Zaluzec et al., 1995; Roepstorff et al., 2000; Nguyen et al., 1995), DNA and oligonucleotide sequencing (Miketova et al., 1997; Faulstich et al., 1997; Bentzley et al., 1996), and the characterization of recombinant proteins (Kanazawa et al., 1999; Villanueva et al., 1999). Recently, applications of MALDI-TOF-MS have been extended to include the direct analysis of biological tissues and single cell organisms with the aim of characterizing endogenous peptide and protein constituents (Li et al., 2000; Lynn et al., 1999; Stoeckli et al., 2001; Caprioli et al., 1997; Chaurand et al., 1999; Jespersen et al., 1999).

The properties that make MALDI-TOF-MS a popular qualitative tool—its ability to analyze molecules across an extensive mass range, high sensitivity, minimal sample preparation and rapid analysis times—also make it a potentially useful quantitative tool. MALDI-TOF-MS also enables non-volatile and thermally labile molecules to be analyzed with relative ease. It is therefore prudent to explore the potential of MALDI-TOF-MS for quantitative analysis in clinical settings, for toxicological screenings, as well as for environmental analysis. In addition, the application of MALDI-TOF-MS to the quantification of peptides and proteins is particularly relevant. The ability to quantify intact proteins in biological tissue and fluids presents a particular challenge in the expanding area of proteomics and investigators urgently require methods to accurately measure the absolute quantity of proteins. While there have been reports of quantitative MALDI-TOF-MS applications, there are many problems inherent to the MALDI ionization process that have restricted its widespread use (Kazmaier et al., 1998; Horak et al., 2001; Gobom et al., 2000; Wang et al., 2000; Desiderio et al., 2000). These limitations primarily stem from factors such as the sample/matrix heterogeneity, which are believed to contribute to the large variability in observed signal intensities for analytes, the limited dynamic range due to detector saturation, and difficulties associated with coupling MALDI-TOF-MS to on-line separation techniques such as liquid chromatography. Combined, these factors are thought to compromise the accuracy, precision, and utility with which quantitative determinations can be made.

Because of these difficulties, practical examples of quantitative applications of MALDI-TOF-MS have been limited. Most of the studies to date have focused on the quantification of low mass analytes, in particular, alkaloids or active ingredients in agricultural or food products (Wang et al., 1999; Jiang et al., 2000; Wang et al., 2000; Yang et al., 2000; Wittmann et al., 2001), whereas other studies have demonstrated the potential of MALDI-TOF-MS for the quantification of biologically relevant analytes such as neuropeptides, proteins, antibiotics, or various metabolites in biological tissue or fluid (Muddiman et al., 1996; Nelson et al., 1994; Duncan et al., 1993; Gobom et al., 2000; Wu et al, 1997; Mirgorodskaya et al., 2000). In earlier work it was shown that linear calibration curves could be generated by MALDI-TOF-MS provided that an appropriate internal standard was employed (Duncan et al., 1993). This standard can “correct” for both sample-to-sample and shot-to-shot variability. Stable isotope labeled internal standards (isotopomers) give the best result.

With the marked improvement in resolution available on modern commercial instruments, primarily because of delayed extraction (Bahr et al., 1997; Takach et al., 1997), the opportunity to extend quantitative work to other examples is now possible; not only of low mass analytes, but also biopolymers. Of particular interest is the prospect of absolute multi-component quantification in biological samples (e.g., proteomics applications).

The properties of the matrix material used in the MALDI method are critical. Only a select group of compounds is useful for the selective desorption of proteins and polypeptides. A review of all the matrix materials available for peptides and proteins shows that there are certain characteristics the compounds must share to be analytically useful. Despite its importance, very little is known about what makes a matrix material “successful” for MALDI. The few materials that do work well are used heavily by all MALDI practitioners and new molecules are constantly being evaluated as potential matrix candidates. With a few exceptions, most of the matrix materials used are solid organic acids. Liquid matrices have also been investigated, but are not used routinely.

D. Immunologic Analysis

As discussed, in some embodiments, the present invention concerns methods for determining ratios of sAPPα/sAPPβ and CTFα/CTFβ. One can use a variety of immunodetection methods to achieve these goals. Such immunodetection methods include enzyme linked immunosorbent assay (ELISA), radioimmunoassay (RIA), immunoradiometric assay, fluoroimmunoassay, chemiluminescent assay, bioluminescent assay, and Western blot, though several others are well known to those of ordinary skill. The steps of various useful immunodetection methods have been described in the scientific literature, such as, e.g., Doolittle et al. (1999); Gulbis et al. (1993); De Jager et al. (1993); and Nakamura et al. (1987), each incorporated herein by reference.

In general, the immunobinding methods include obtaining a sample suspected of containing a protein, polypeptide and/or peptide, and contacting the sample with a first antibody, monoclonal or polyclonal, in accordance with the present invention, as the case may be, under conditions effective to allow the formation of immunocomplexes.

These methods include methods for purifying a protein, polypeptide and/or peptide from organelle, cell, tissue or organism's samples. In these instances, the antibody removes the antigenic protein, polypeptide and/or peptide component from a sample. The antibody will preferably be linked to a solid support, such as in the form of a column matrix, and the sample suspected of containing the protein, polypeptide and/or peptide antigenic component will be applied to the immobilized antibody. The unwanted components will be washed from the column, leaving the antigen immunocomplexed to the immobilized antibody to be eluted.

The immunobinding methods also include methods for detecting and quantifying the amount of an antigen component in a sample and the detection and quantification of any immune complexes formed during the binding process. Here, one would obtain a sample suspected of containing an antigen or antigenic domain, and contact the sample with an antibody against the antigen or antigenic domain, and then detect and quantify the amount of immune complexes formed under the specific conditions.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection designed by Charles Cantor uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

As detailed above, immunoassays, in their most simple and/or direct sense, are binding assays. Certain preferred immunoassays are the various types of enzyme linked immunosorbent assays (ELISAs) and/or radioimmunoassays (RIA) known in the art. Immunohistochemical detection using tissue sections is also particularly useful. However, it will be readily appreciated that detection is not limited to such techniques, and/or western blotting, dot blotting, FACS analyses, and/or the like may also be used.

In one exemplary ELISA, antibodies are immobilized onto a selected surface exhibiting protein affinity, such as a well in a polystyrene microtiter plate. Then, a test composition suspected of containing the antigen, such as a clinical sample, is added to the wells. After binding and/or washing to remove non-specifically bound immune complexes, the bound antigen may be detected. Detection is generally achieved by the addition of another antibody that is linked to a detectable label. This type of ELISA is a simple “sandwich ELISA.” Detection may also be achieved by the addition of a second antibody, followed by the addition of a third antibody that has binding affinity for the second antibody, with the third antibody being linked to a detectable label. The ELISA may be based on differential binding of an antibody to a protein with Arg389 versus Gly389.

In another exemplary ELISA, the samples suspected of containing the antigen are immobilized onto the well surface and/or then contacted with antibodies. After binding and/or washing to remove non-specifically bound immune complexes, the bound anti-antibodies are detected. Where the initial antibodies are linked to a detectable label, the immune complexes may be detected directly. Again, the immune complexes may be detected using a second antibody that has binding affinity for the first antibody, with the second antibody being linked to a detectable label.

Another ELISA in which the antigens are immobilized, involves the use of antibody competition in the detection. In this ELISA, labeled antibodies against an antigen are added to the wells, allowed to bind, and/or detected by means of their label. The amount of an antigen in an unknown sample is then determined by mixing the sample with the labeled antibodies against the antigen during incubation with coated wells. The presence of an antigen in the sample acts to reduce the amount of antibody against the antigen available for binding to the well and thus reduces the ultimate signal. This is also appropriate for detecting antibodies against an antigen in an unknown sample, where the unlabeled antibodies bind to the antigen-coated wells and also reduces the amount of antigen available to bind the labeled antibodies.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. An example of a washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. This may be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

For example, measurement of α-CTF and β-CTF can be performed by western blot. The pelleted cells will be washed with 1 ml of PBS and centrifuged at 15,000×g for 10 min. The cells will then be lyzed by sonication in 200 μl of PBS containing protease inhibitors and protein concentration will be determined using bicinconinic acid (BCA, Pierce). Cell lysates for each sample (50 μg protein) will be subjected to SDS-PAGE and the proteins will be transferred to nitrocellulose membranes. Membranes will then be blocked for 2 h with 10% fat-free milk in PBS and incubated with a C-terminal anti-APP antibody (A8717, Sigma) at a 1:2000 dilution over night at 4° C. α- and β-CTF will then be detected using a goat anti-rabbit IgG conjugated with horseradish peroxidase, at a 1:10000 dilution for 1 hour at room temperature. Bands will be resolved using chemiluminescence (ECL, Pierce).

E. Exemplary sAPP Protocol

Immunoprecipitation of sAPPs. sAPPs will be immunoprecipitated in CSF samples using a mouse anti-APP monoclonal antibody (NBA-100, Stressgen Bioresearch) that recognizes an epitope at the N-terminal end of APP. Approximately 2 ml of CSF have been shown to be sufficient for providing sAPPs for the analysis by LC/ESI/MS/MS. Thus, CSF (2 ml) will be diluted with 3 volumes of PBS and incubated with 6 mg of agarose beads conjugated to the antibody overnight at 4° C. on a rocker. Pelleted beads will be washed extensively in PBS. The beads-IgG-APP complex will be used for tryptic digestion as described below.

Immunoprecipitation of sAPPs from frozen tissues. sAPPα/sAPPβ ratio will can also be measured in pieces of frozen cortex and pieces of frozen cerebellum. The frozen tissues will be thawed and homogenized in 3 volumes of PBS containing a protease inhibitor cocktail. The homogenate will be centrifuged at 10,000×g for 15 min at 4° C. to remove cellular debris. The supernatant will be centrifuged at 100,000×g for 90 min at 4° C. The supernatant will be used to immunoprecipitate sAPPs as described above. The pellet will be resuspended in 1 volume of PBS corresponding to the initial weight of the piece of tissue.

Digestion of immunoprecipitated sAPPs. sAPPs will be digested using procedures and reagents for tryptic digestion of proteins described previously (Manza et al., 2005). In short, the pelleted beads will be resuspended in 30 μl of 100 mM ammonium bicarbonate containing 1 Unit of modified trypsin (Promega, Madison, Wis.) and incubated at 37° C. overnight. The beads will be removed by centrifugation and the supernatant containing the tryptic peptides will be collected. Then, 150 pg of MDAE[¹³C]-FR and TEE[¹³C]-ISEVK will be added as internal standards and the samples will be analyzed by LC/ESI/MS/MS, as described below.

Digestion of sAPPs in biological fluids. SAPPs will be digested directly in CSF without purification. Five μl of ambic buffer 1 M will be added to 50 μl of CSF. 2.5 U of trypsin (Trypsin Gold, Promega, Madison, Wis.) will be added and incubated at 37° C. for 24 h. Another 2.5 U of trypsin will be added at this time and incubated at 37° C. for 24 h. Then, 100 PG of MDAE[¹³C]-FR and TEE[¹³C]-ISEVK will be added as internal standards and the samples will be analyzed by LC/ESI/MS/MS.

Analysis of proteolytic peptides by LC/ESI/MS/MS. The digest will be analyzed by LC/ESI/MS/MS. Samples will be analyzed by automated reverse-phase LC/MS using the LCQ Deca XP ion trap mass spectrometer (Thermo Scientific, Waltham, Mass.). UPLC separation of the tryptic peptides will be achieved with Acquity UPLC BEH130 C18 1.7 μm 2.1×100 column (Waters), at 0.4 ml/min flow rate. The peptides will be injected by an autosampler, and separated using an acetonitrile gradient. Solvent A will be 2% acetonitrile with 0.2% formic acid, and solvent B will be 95% acetonitrile containing 0.2% formic acid. The gradient program will be: 0-0.5 min 100% A, 0.5-6.5 min linear gradient from 0% to 20% B; 6.5-7 min, linear gradient from 20% to 100% B; 7-7.5 min 100% B; 7.5-8 min, linear gradient to 100% A; 8-11 min 100% A. The MS/MS spectra of the peptides will be performed in a targeted fashion by selecting specific masses for MS/MS fragmentation using an isolation width of 2 m/z, normalized collision energy of 30%, isolation Q of 0.250 and activation time of 30 ms. The mass spectrometer will be tuned prior to analysis using the synthetic peptides TEEISEVK and MDAEFR. Ion chromatograms will be extracted using the Xcalibur program.

Quantitation of Target Peptides by NanoFlow LC-MS/MS. Preliminary experiments indicate that the inventor can detect as low as 4.5 pg of peptides in CSF. However, in order to increase the sensitivity of the assay, alternative instruments for the analysis of the peptides in biological fluids will be assessed. LC-MS analysis of the tryptic peptides will be performed using a Thermo LTQ linear ion trap mass spectrometer equipped with a Thermo MicroAS autosampler, Thermo Surveyor HPLC pump, and Nanospray ion source using instruments provided by the Vanderbilt University Proteomics Core. Data acquisition will be done using the Xcalibur 2.0 instrument control software. The crude peptides from trypsin digestion of APP will be isolated and partially purified using an inline solid phase extraction column (100 μm ID×6 cm) packed with a C₁₈ resin (Jupiter C₁₈, 5 μm dₚ, 300 Å, Phenomonex, Torrance, Calif.), similar to methods previously described (Licklider et al., 2002). The peptides will be further separated on a capillary tip (100 μm×11 cm) packed with the same C₁₈ resin and eluted using a 0.1% formic acid/acetonitrile gradient. Nanoflow LC/MS/MS typically achieves about a 100-fold improvement in sensitivity compared to conventional low-flow HPLC columns (Karlsson et al., 2004).

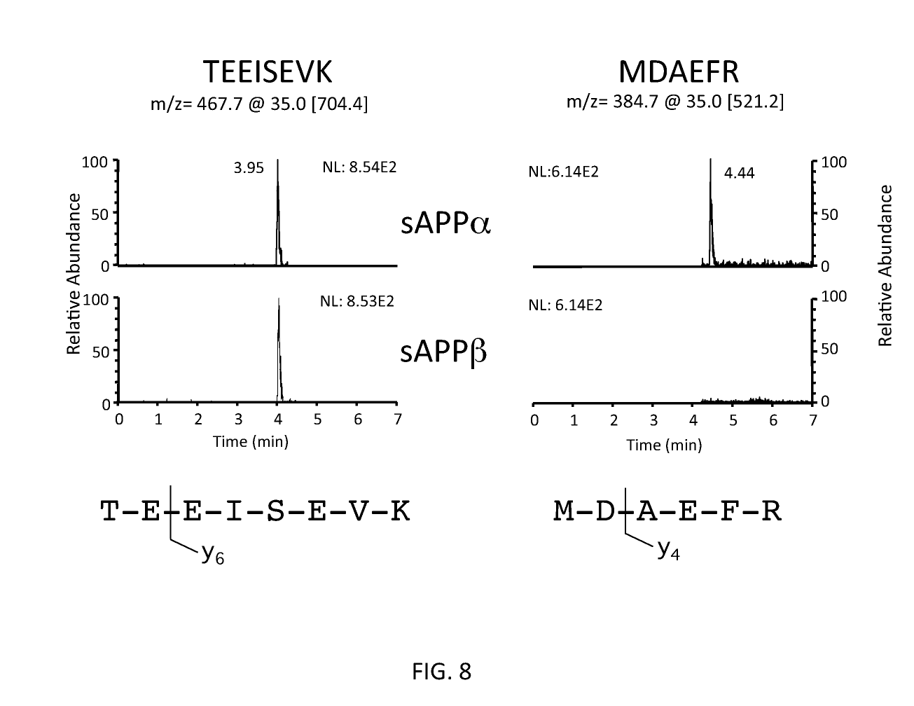

Quantitation will be achieved by selected reaction monitoring (SRM) of the two major product ions from both TEEISEVK and MDAEFR, as shown in FIG. 8. Exact amounts of both peptides within each sample will be measured using the internal standards. The ratio of the two amounts will then be calculated. This method retains the advantage of looking simultaneously at the products of both secretase pathways in the same sample. However, it provides the advantage of quantifying the absolute values of both peptides present using an isotopically labeled internal standard. Because the internal standard is identical to the product of digestion and because it will be subjected to the same isolation and ionization procedures, it will automatically correct for any impairment in the chromatographic and/or ionization processes. The amounts of peptides will be calculated using the known amount of internal standard as reference, similar to what has been previously done with the levuglandinyl-lysyl lactam adducts (Zagol-Ikapitte et al., 2005).

F. Additional Diagnostic Approaches

In various aspects of the invention, it will be desirable to further subject patients to more traditional AD diagnostic approaches. Such general approaches for diagnosis are set out below.

The diagnosis of both early (mild) cognitive impairment and AD are based primarily on clinical judgment. However, a variety of neuropsychological tests aid the clinician in reaching a diagnosis. Early detection of only memory deficits may be helpful in suggesting early signs of AD, since other dementias may present with memory deficits and other signs. Cognitive performance tests that assess early global cognitive dysfunction are useful, as well as measures of working memory, episodic memory, semantic memory, perceptual speed and visuospatial ability. These tests can be administered clinically, alone or in combination. Examples of cognitive tests according to cognitive domain are shown as examples, and include “Digits Backward” and “Symbol Digit” (Attention), “Word List Recall” and “Word List Recognition” (Memory), “Boston Naming” and “Category Fluency” (Language), “MMSE 1-10” (Orientation), and “Line Orientation” (Visuospatial). Thus, neuropsychological tests and education-adjusted ratings are assessed in combination with data on effort, education, occupation, and motor and sensory deficits. Since there are no consensus criteria to clinically diagnose mild cognitive impairment, various combinations of the above plus the clinical examination by an experienced neuropsychologist or neurologist are key to proper diagnosis. As the disease becomes more manifest (i.e., becomes a dementia rather than mild cognitive impairment), the clinician may use the criteria for dementia and AD set out by the joint working group of the National Institute of Neurologic and Communicative Disorders and Stroke/AD and Related Disorders Association (NINCDS/ADRDA). On occasion, a clinician may request a head computed tomography (CT) or a head magnetic resonance imaging (MRI) to assess degree of lobar atrophy, although this is not a requirement for the clinical diagnosis.

### IV. Therapies and Assessment Thereof

A. Treatment Efficacy

Various drugs for the treatment of AD are currently available as well as under study and regulatory consideration. The drugs generally fit into the broad categories of cholinesterase inhibitors, muscarinic agonists, anti-oxidants or anti-inflammatories. Galantamine (Reminyl), tacrine (Cognex), selegiline, physostigmine, revistigmin, donepezil, (Aricept), rivastigmine (Exelon), metrifonate, milameline, xanomeline, saeluzole, acetyl-L-carnitine, idebenone, ENA-713, memric, quetiapine, neurestrol and neuromidal are just some of the drugs proposed as therapeutic agents for AD.

The present invention contemplate use of the “diagnostic” procedures, discussed above, to further assess the efficacy of treatments. Given the role of Aβ in AD, the ability of a particular therapy to reduce the amount of Aβ will be indicative of an effective treatment, since low amounts of Aβ will presumably reduce the rate or development of plaques, thereby impeding the progression of the disease.

B. Prostanoid Receptor Agonists and Antagonists

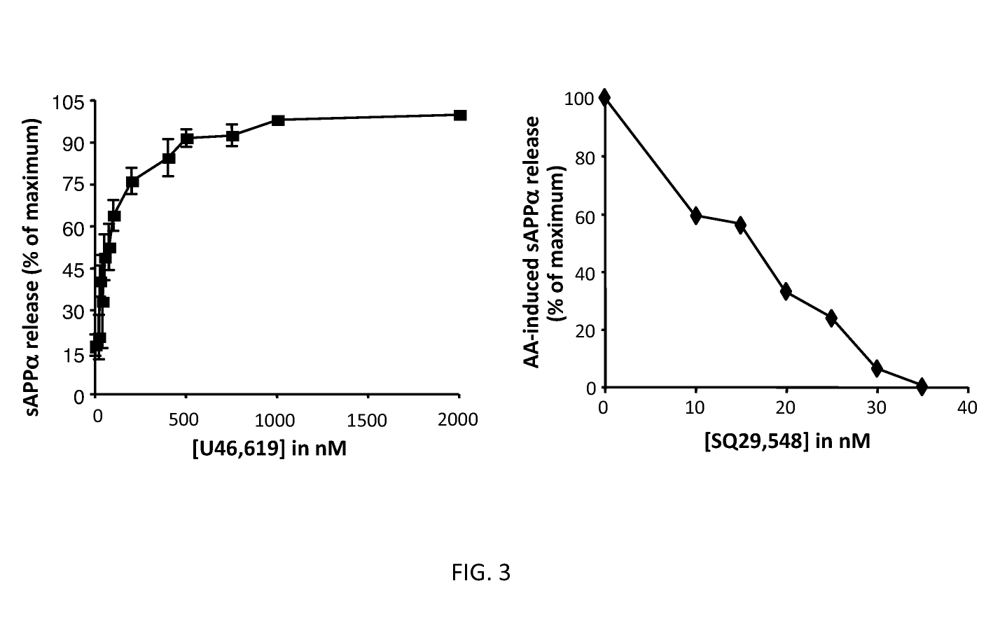

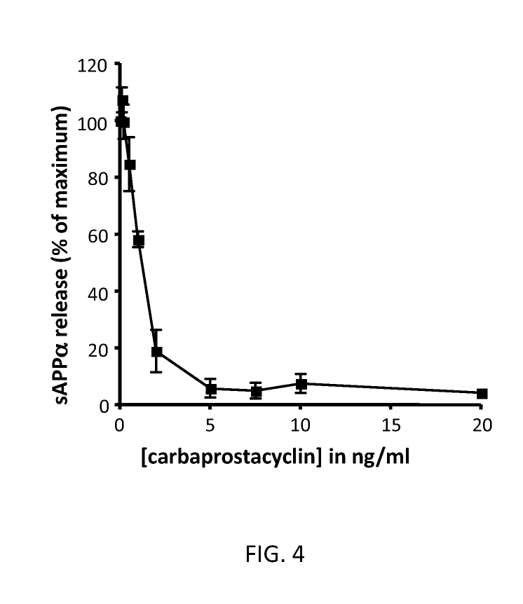

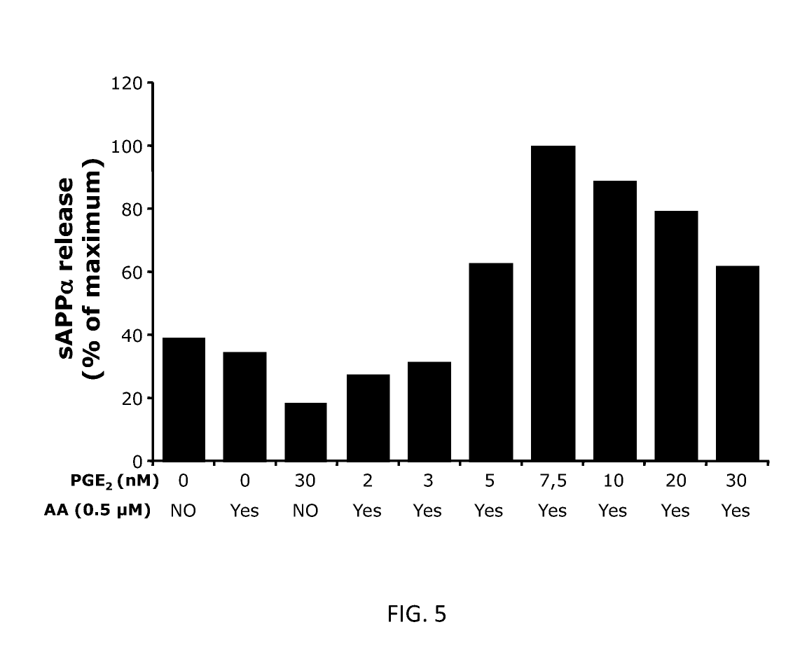

Preliminary data indicates that agonists of the thromboxane receptor or of some of the isoforms of the prostaglandin E₂ receptors can increase the α-secretase activity (see FIGS. 3 and 5). In contrast, an agonist of the prostaglandin I₂ (prostacyclin) receptor would lead to the opposite effect (see FIG. 4). Accordingly, any drug with agonist, partial agonist or allosteric activation properties of the TP or any isoform of the EP receptors could be used to activate the α-secretase activity with potential therapeutic effect on AD. Examples of such agonists include CP-533,536, CP-536, 745-01, and 17-phenyl-trinor-PGE₂. Other prostaglandin E2 receptor agonists are described in U.S. Pat. Nos. 5,663,417, 6,046,236 and 7,091,231 and in U.S. Patent Publication No. 20060148894. Similarly, if an increase in prostacyclin signaling in the brain is associated with the observed decreases α-secretase activity, a antagonist or allosteric modulator of the prostaglandin I₂ receptor could be used to lift this inhibition and restore normal α-secretase activity. PGI₂ antagonists are described in WO 2005/005394.

C. Pharmaceutical Compositions and Routes of Delivery

The present invention may involve the use of pharmaceutical compositions which comprise an agent, such as discussed above, and a pharmaceutically acceptable carrier. As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18^{th} Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The compositions of the present invention may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostatically, intrapleurally, intranasally, intramuscularly, intraperitoneally, subcutaneously, mucosally, orally, topically, inhalation (e.g., aerosol inhalation), by injection, infusion, continuous infusion, in lipid compositions (e.g., liposomes), or by other method or any combination of the foregoing as would be known to one of ordinary skill in the art (see, for example, Remington's Pharmaceutical Sciences, 18^{th} Ed. Mack Printing Company, 1990, incorporated herein by reference).

The actual dosage amount of a composition of the present invention administered to a patient can be determined by physical and physiological factors such as body weight, severity of disease, previous or concurrent therapeutic interventions, idiopathy of the patient and on the route of administration. The practitioner responsible for administration will, in any event, determine the concentration of active ingredient(s) in a composition and appropriate dose(s) for the individual subject.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The compounds of the present invention may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In certain embodiments the compounds of the present invention are prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein. In particular embodiments, prolonged absorption of an injectable composition can be brought about by the use in the compositions of agents delaying absorption, such as, for example, aluminum monostearate, gelatin or combinations thereof.

### V. Screening Methods

The present invention further comprises methods for screening of drugs for modulators of sAPPα to sAPPβ or αCTF to βCTF ratios. These assays may comprise random screening of large libraries of candidate substances; alternatively, the assays may be used to focus on particular classes of compounds selected with an eye towards attributes that are believed to make them more likely to provide the desired results.

A. Candidate Substances

As used herein the term “candidate substance” refers to any molecule that may potentially increase sAPPα to sAPPβ or αCTF to βCTF ratios. The candidate substance may be a peptide, polypeptide, a small molecule, or even a nucleic acid molecule.

Using lead compounds to help develop improved compounds is known as “rational drug design.” The goal of rational drug design is to produce structural analogs of compounds and systematically test these to arrive at suitable compounds with the desired properties. In one approach, one would generate a three-dimensional structure for a target molecule, or a fragment thereof. This could be accomplished by x-ray crystallography, computer modeling or by a combination of both approaches. In another approach, the structural analogs can be modeled after the predicted primary, secondary or tertiary structure of a protein or peptide.

It also is possible to use antibodies to ascertain the structure of a target compound activator or inhibitor, thereby bypassing protein crystallography altogether by generating anti-idiotypic antibodies. As a mirror image of a mirror image, the binding site of anti-idiotype would be expected to be an analog of the original antigen. The anti-idiotype could then be used to identify and isolate compounds from banks of chemicals or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for a target molecule and expressed in a cell that expresses Aβ or a precursor thereof.

It will, of course, be understood that all the screening methods of the present invention are useful in themselves notwithstanding the fact that effective candidates may not be found. The invention provides methods for screening for such candidates, not solely methods of finding them.

B. In vitro Assays

To identify an agent that increase sAPPα to sAPPβor αCTF to βCTF ratios, one generally will determine these ratios in the presence and absence of the candidate substance. For example, a method generally comprises:
(a) providing a candidate substance;
(b) contacting the candidate with a cell that expresses sAPP and/or CTF;
(c) measuring a sAPPα to sAPPβ or αCTF to βCTF ratio; and
(d) comparing the ratio measured in step (c) with the ratio in the absence of the candidate modulator,
   wherein an increase in the sAPPα to sAPPβor αCTF to βCTF ratio indicates that the candidate modulator inhibits Aβ production.

Depending on the assay, culture may be required. The cell may be examined using any of a number of different assays, as discussed above. Suitable cells include a neuron, platelets, microglia or astrocyte, or cell lines derived therefrom.

C. In vivo Assays

In accordance with the present invention, one may also assess sAPPα to sAPPβ or αCTF to βCTF ratios in vivo following treatment with candidates, alone or in combination with other drugs. In an exemplary assay, an agent is provided to an experimental animal via an appropriate route. sAPPα to sAPPβor αCTF to βCTF ratios are then assessed and compared to those seen in a similar animal not receiving the agent, e.g., the same animal prior to receiving the inhibitor or a similar (control) animal. The assessment may also comprise various other parameters, including timing of administration, varying the dose, assessing toxicity.

Mouse models with clinical features suggestive of AD have been generated. The amyloid β (A4) precursor protein (APP) targeted mutation mice were generated by Dr. David Borchelt and can be purchased from The Jackson Laboratory (Bar Harbor, Me.). This mouse model develops decreased forelimb grip strength and locomotor activity. In addition, reactive astrocytosis can be demonstrated by histopathology by 14 weeks of age. The double transgenic APP (chimeric-mouse/human)-presenilin 1 (human), also generated by Dr. David Borchelt, can also be obtained from the Jackson Laboratory. The latter mice start accumulating amyloid deposits in the brain by nine months of age, similar to those found in human AD brains. These deposits increase dramatically by age 12 months. AD mouse models develop behavioral alterations that can be assessed using various tests, including the water maze, T maze, or contextual fear conditioning tests. Thus, a drug proposed to ameliorate AD in humans can be assessed and validated on the AD animal models. Other AD mice with various levels of expression of APPs have been generated, including animals that develop signs of disease or synaptic toxicity prior to plaque formation (Mucke et al., 2000). Models with the various mutations leading to AD-like pathology are reviewed in Price and Sisodia (1998). This model will find use in screening of compounds according to the present invention for activity against AD and symptoms thereof.

### VI. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Pharmacologic Regulation of sAPPα Release by Human Platelets

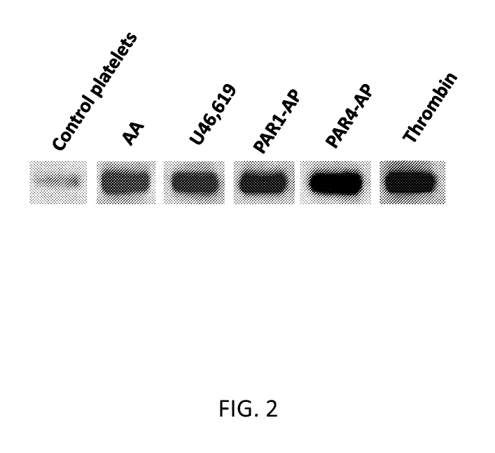

Secretion of sAPPα has been analyzed in human platelets in response to various agonists. As shown in FIG. 2, activation of the TP receptor with a specific agonist U46,619 gave the same response as 20 μM of arachidonic acid, suggesting that the effect of the fatty acid was mediated by thromboxane. When using agonists specific for the two protease-activated receptors (PAR) known to be present in human platelets, PAR1 and PAR4, similar levels of release of sAPPα were observed as with thrombin, indicating that PARs also participate in sAPPα release (FIG. 2).

Experiments were performed to determine the optimal concentrations of arachidonic acid and of U46,619 for use in latter experiments. The maximal effect was obtained with 7.5 μM of arachidonic acid and this concentration is now used in all experiments. As seen in FIG. 3, the maximal response was obtained with 500 nM of TP receptor agonist. This concentration is now used in every experiment unless otherwise mentioned. The inventor showed that the effect of 7.5 μM of arachidonic acid was antagonized by SQ29,548, a specific antagonist of the TP receptor, in a dose-dependent manner (FIG. 3, right panel), confirming that the effect of arachidonic acid was mediated through the TP receptor.

Interestingly, doses of collagen up to 5 μg/ml that cause full platelet aggregation and dense-granule release (data not shown), failed to trigger release of sAPPα by washed platelets. Similarly, incubation of washed platelets with ADP up to 25 μM had no effect on sAPPα release. These results suggest the possibility to characterize a transduction pathway specific for up-regulating α-secretase.

To further characterize the pharmacology of sAPPα release, the inventor investigated the effects of agonists of the other prostaglandin receptors. They obtained results indicating that carbaprostacyclin (CP), a stable analog of prostacyclin, antagonized in a dose-dependent manner the effect of U46,619 (FIG. 4). These results suggest that α-secretase up-regulation is mediated by receptors signaling through calcium release and is antagonized by receptors increasing cAMP.

Recent evidence suggest that PGE₂ can potentiate platelet aggregation in mice when sub-efficacious doses of agonists are used, and this effect is thought to be mediated by the EP3 receptor (Fabre et al., 2001; Gross et al., 2007). Accordingly, the inventor tested PGE₂ as an activator of α-secretase and found that at low nanomolar concentrations, PGE₂ also potentiates sAPPα release from human platelet (FIG. 5). However, at concentrations higher than 10 nM, this potentiation effect was reversed, suggesting the possibility that different receptor sub-types may be involved in this mechanism. Importantly, the highest concentration of PGE₂ used by itself had no effect on sAPPα release, supporting the hypothesis that G_{q} does not mediate PGE₂ signaling.

Taken together, these preliminary results indicate that α-secretase activity is strongly regulated by prostaglandin receptors that can either up-regulate or down-regulate its catalytic activity.

### Example 2

### Analysis of the Ratios of sAPPα/sAPPβ as a Marker of α-Secretase Regulation

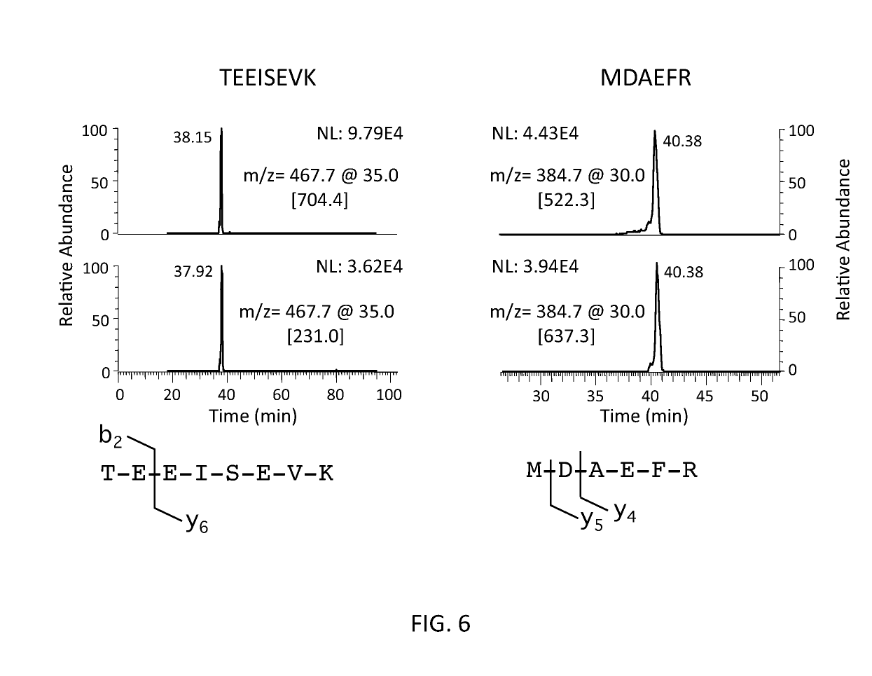

The inventor developed a mass spectrometry method for assessing sAPP. Two hundred ng of human recombinant sAPPα and sAPPβ were digested in solution with trypsin (Trypsin Gold, Promega, Madison, Wis.)) as previously described (Manza et al., 2005). LC-MS analysis of the resulting peptides was performed using a ThermoFinnigan LTQ ion trap mass spectrometer. Ion chromatograms were extracted using the Xcalibur program by selecting the MS/MS events and associating them with particular fragment ions to produce traces similar to what would be seen with a selected reaction monitoring experiment to see which fragment ions produced the cleanest traces with the greatest abundance. sAPPα and sAPPβdiffer only by the C-terminal sequence: sAPPα contains 16 extra amino acids versus sAPPβ. Therefore, the inventor focused his attention on the analysis of peptides around the β-secretase cleavage site. Accordingly, two peptides were selected after analysis of the spectra (FIG. 6).

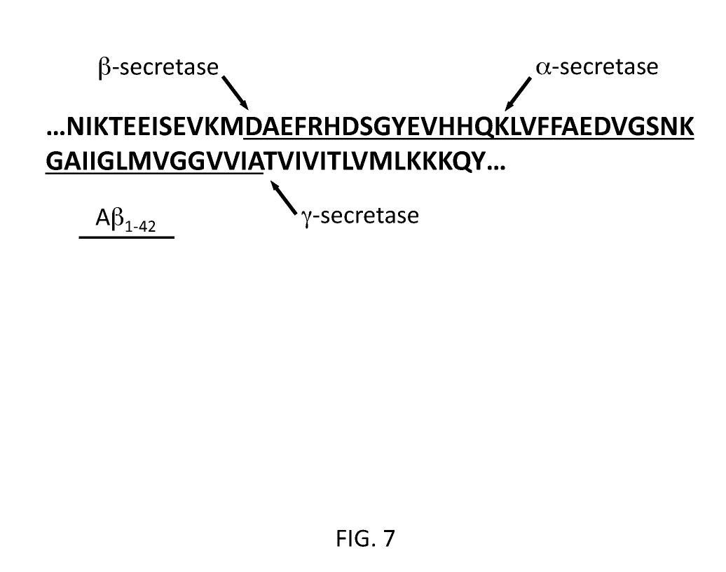

Analysis of the mass spectra of the peptide TEEISEVK determined that there was a prominent cleavage after amino acid 2, yielding the y2 and b6 ions (FIG. 7). Cleavages after the amino acids 1 and 2 of MDAEFR, yielded the y4 and y5 ions, respectively (FIG. 7). The inventor then assessed the specificity of the two peptides for the respective sAPP isoforms. As shown in FIG. 8, the TEEISEVK peptide is present in both sAPPα and sAPPβand can be used as an indicator of the total sAPP present in the sample. In contrast, the MDAEFR peptide is only generated by digestion of sAPPα (FIG. 8).

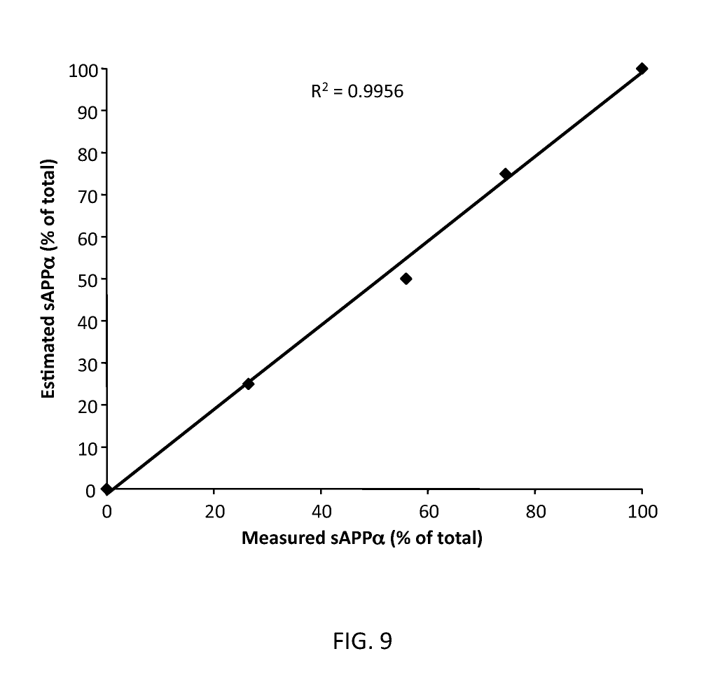

Using these products ions, the inventor analyzed samples containing varying ratios of sAPPα/sAPPβmixed in 10% of fetal beef serum (FBS). After isolating the sAPPs by immunoprecipitation, the proteins were digested by trypsin and the peptides were analyzed by LC/ESI/MS/MS. Then, he calculated the relative abundance of MDAEFR over TEEISEVK and found that the ratios of the areas for the different product ions correlated with the ratios of sAPPs present in the samples (FIG. 9). These results indicate that the assay is suitable for the experiments proposed in this application.

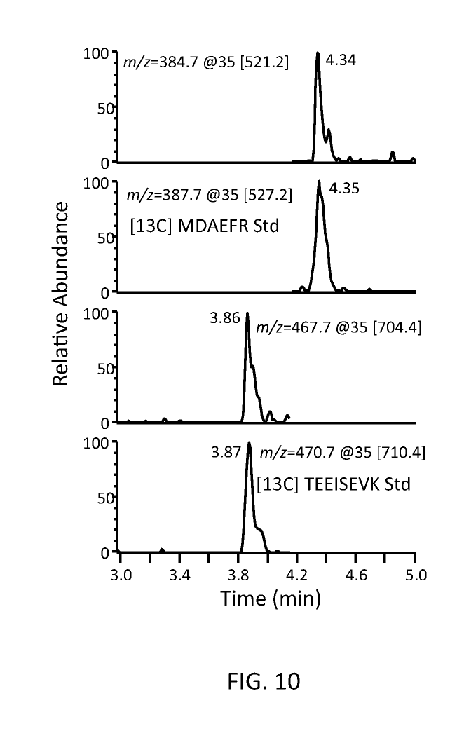

Internal standards for each peptide were obtained by incorporation of a [¹³C] labeled amino acid. Human washed platelets (600,000 cells/Ml, 300 μl) were activated with 500 nM of U46,619 and the release of sAPPs was analyzed after centrifuging the cells at 2,000×g for 15 min. After immunoprecipitation of the sAPPs and digestion with trypsin, 150 pg of MDAE[¹³C]-FR and TEE[¹³C]-ISEVK were added as internal standards and the samples were analyzed by LC/ESI/MS/MS using the LCQ Deca XP ion trap mass spectrometer (Thermo Scientific, Waltham, Mass.). The results indicate that, in these conditions, platelets release equal amounts of sAPPα and sAPPβ(45 and 41 pg, respectively) (FIG. 10).

The same method was employed to measure the relative abundances of sAPPα to sAPPβ in CSF from patients with confirmed diagnostic of AD and age-matched controls. Between 1.2 and 2 ml of CSF were diluted with 3 volumes of PBS, and the sAPPs were immunoprecipitated and digested with trypsin as described above. After adding 150 pg of deuterated internal standards, the samples were analyzed by mass spectrometry (FIG. 1).

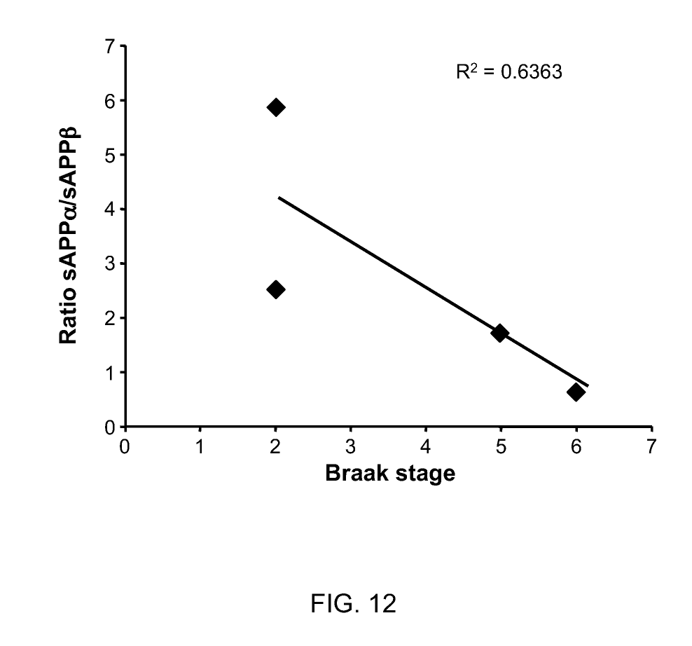

The amount of sAPPs present in the samples was determined by isotopic dilution and the ratios of sAPPα/sAPPβ were calculated for each samples. The relative abundance of sAPPα to sAPPβ was found to be inversely proportional to the Braak stage, one of the therapeutic indexes of AD (FIG. 12).

### Example 3

### Validation and Reproducibility of sAPPα/sAPPβ Ratios in CSF and Platelets

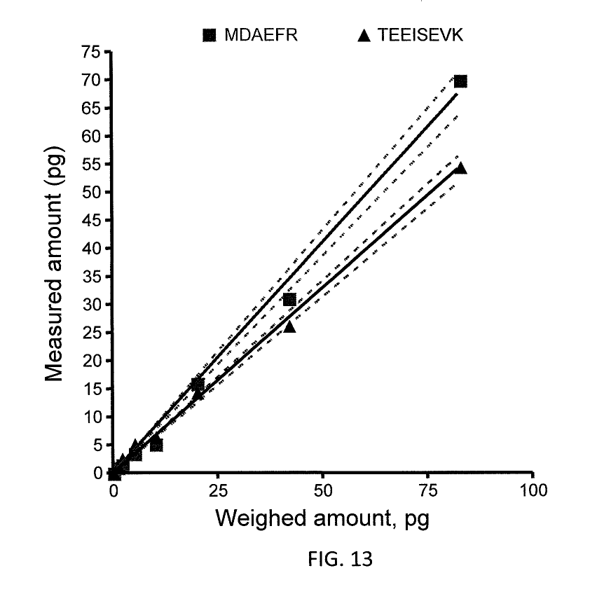

The inventor performed a series of experiments aimed at assessing the reproducibility of and at validating the mass spectrometric method. Varying amounts of synthetic peptides (MDAEFR and TEEISEVK) were weighed and diluted in ammonium bicarbonate buffer (ambic) 100 mM containing 1% lysine (w/v). Then, 100 PG of MDAE[¹³C]-FR and TEE[¹³C]-ISEVK were added as internal standard and the samples were analyzed by LC/ESI/MS/MS. The amount of synthetic peptides present in each sample was determined by isotopic dilution. As shown in FIG. 13, the mass spectrometic signal is linear within the range of concentrations of peptides present in biological samples.

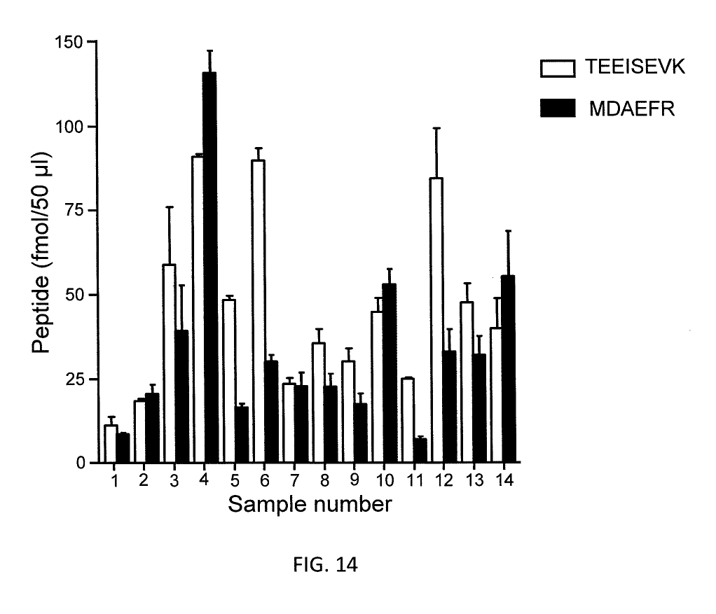

The reproducibility of the assay for CSF is shown in FIG. 14. The amount of peptides (MDAEFR and TEEISEVK) were measured in the same human CSF samples three different times. Five μl of ambic buffer 1 M was added to 50 μl of CSF. 2.5 U of trypsin (Trypsin Gold, Promega, Madison, Wis.) was added and incubated at 37° C. for 24 h. Another 2.5 U of trypsin was added at this time and incubated at 37° C. for 24 h. Then, 100 PG of MDAE[¹³C]-FR and TEE[¹³C]-ISEVK were added as internal standards and the samples were analyzed by LC/ESI/MS/MS. This experiment demonstrate good reproducibility of the assay in CSF.

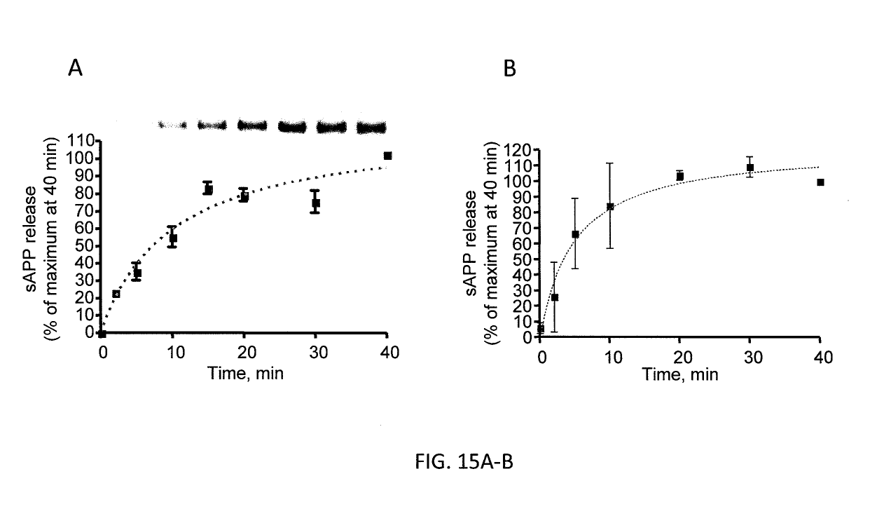

To assess whether the LC/ESI/MS/MS method can be used to monitor α-secretase activity, human platelets were activated with an agonist of the thromboxane receptor (U46,619). At different times, the platelets were centrifuged and the supernatant was aliquoted in two fractions. One fraction was used to analyze the release of sAPPα by western blot (FIG. 15A) and the other aliquot was used to analyzed sAPPα by LC/ESI/MS/MS (FIG. 15B). The results demonstrate a good correlation between the two methods, validating the LC/ESI/MS/MS method to measure sAPPs.

The reproducibility of the LC/ESI/MS/MS assay for platelets was assessed by repeating the experiment described above 3 independent times. After activating the platelets with 500 nM U46,619, the platelets were centrifuged and the released sAPP in the supernatant was digested two times for 24 h with 2.5 U of trypsin (Trypsin Gold, Promega, Madison, Wis.) at 37° C. Then, 100 PG of MDAE[¹³C]-FR and TEE[¹³C]-ISEVK were added as internal standards and the samples were analyzed by LC/ESI/MS/MS. This experiment demonstrate good reproducibility of the assay for human platelets.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VII. References

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,275,149
U.S. Pat. No. 4,277,437
U.S. Pat. No. 4,366,241
U.S. Pat. No. 5,663,417
U.S. Pat. No. 5,757,994
U.S. Pat. No. 5,788,166
U.S. Pat. No. 5,838,002
U.S. Pat. No. 5,986,258.
U.S. Pat. No. 6,046,236
U.S. Pat. No. 7,091,231
U.S. Pat. RE 35,413
U.S. Patent Publn. 2006/0148894
Adie et al., Biochem. J., 2852):529-536, 1992.
Bahr et al., J. Mass Spectrom., 32:1111-1116, 1997.
Bentzley et al., Anal Chem., 68(13):2141-2146, 1996.
Bucknall et al., J. Am. Soc. Mass Spectrom., 13(9):1015-1027, 2002.
Buxbaum et al., J. Biol. Chem., 273:27765-27767, 1998.
Caccamo et al., Neuron., 49:671-682, 2006.
Capaldi et al., Biochem. Biophys. Res. Comm., 74(2):425-433, 1977.
Caprioli et al., Anal. Chem., 69:4751, 1997.
Chaurand et al., Anal Chem., 71(23):5263-5270, 1999.
Chen et al., Nat. Biotechnol., 19:537-542, 2001.
Colciaghi et al., Mol. Med., 8:67-74, 2002.
Consensus report of the Working Group on: “Molecular and Biochemical Markers of Alzheimer's Disease”, Neurobiol. Aging, 19:109-116, 1998.
De Jager et al., Semin. Nucl. Med., 23(2):165-179, 1993.
De Strooper and Annaert, J. Cell Sci. 113(11):1857-1870, 2000.
Desiderio et al., J. Mass Spectrom., 35(6):725-733, 2000.
Desiderio et al., Methods Mol. Biol., 61:57-65, 1996.
Djellas et al., J. Biol. Chem., 274:14325-14330, 1999.
Doolittle and Ben-Zeev, Methods Mol, Biol, 109:215-237, 1999.
Duncan et al., Rapid Commun. Mass Spectrom., 7(12):1090-1094, 1993.
Fabre et al., J. Clin. Invest., 107:603-610, 2001.
Fagan et al., J. Alzheimers Dis., 8:347-358, 2005.
Faulstich et al., Anal Chem., 69(21):4349-4353, 1997.
Galasko, J. Alzheimers Dis., 8:339-346, 2005.
Gilles et al., J. Biol. Chem., 254(5):1462-1468, 1979.
Gobom et al., Anal. Chem., 72(14):3320-3326, 2000.
Gross et al., J. Exp. Med., 204:311-320, 2007.
Gulbis and Galand, Hum. Pathol., 24(12):1271-1285, 1993.
Hebert et al., Am. J. Physiol., 259:F318-325, 1990.
Honda et al., J. Biol. Chem., 268:7759-7762, 1993.
Hooper and Turner, Curr. Med. Chem., 9:1107-1119, 2002.
Horak et al., Rapid Commun. Mass Spectrom., 15(4):241-248, 2001.
Jespersen et al., Anal Chem., 71(3):660-666, 1999.
Jiang et al., Biochem. Pharmacol., 59:763-772, 2000.
Kabarle et al., Anal. Chem. 65(20):972A-986A, 1993.
Kanazawa et al., Biol. Pharm. Bull., 22(4):339-346, 1999.
Kang et al., Nature, 325:733-736, 1987.
Karlsson et al., Rapid. Commun. Mass Spectrom., 18:2282-2292, 2004.
Katoh et al., J. Biol. Chem., 273:2489-2492, 1998.
Kazmaier et al., Anesthesiology, 89(4):831-817, 1998.
King and Turner, J. Neurochem., 88(4):971-982, 2004.
Kinsella et al., J. Pharmacol. Exp. Ther., 281:957-964, 1997.
Kojro and Fahrenholz, Subcell. Biochem., 38:105-127, 2005.
Kojro et al., FASEB J., 20:512-514, 2006.
Kojro et al., Proc. Natl. Acad. Sci. USA, 98:5815-5820, 2001.
Lehner et al., J. Leukoc. Biol., 83(4):883-893, 2008.
Li et al., Proc. Natl. Acad. Sci. USA, 97(4):1566-1571, 2000.
Liang et al., J. Neurosci., 25:10180-10187, 2005.
Licklider et al., Anal. Chem., 74:3076-3083, 2002.
Lovelace et al., J. Chromatogr., 562(1-2):573-584, 1991.
Lynn et al., J. Mol. Evol., 48(5):605-614, 1999.
Manza et al., Proteomics, 5:1742-1745, 2005.
Marie et al., Anal. Chem., 72(20):5106-5114, 2000.
Miketova and Schram, Mol. Biotechnol., 8(3):249-253, 1997.
Mirgorodskaya et al., Rapid Commun. Mass Spectrom., 14(14):1226-1232, 2000.
Mucke et al., J. Neuroscience 20: 4050-4058, 2000.
Muddiman et al., Fres. J. Anal. Chem., 354:103, 1996.
Nakamura et al., In: Handbook of Experimental Immunology (4^{th} Ed.), Weir et al. (Eds), 1:27, Blackwell Scientific Publ., Oxford, 1987.
Nelson et al., Anal. Chem., 66:1408, 1994.
Nguyen et al., Genomics, 29:207-216, 1995.
Nishihara et al., Jpn. J. Pharmacol., 82:226-231, 2000.
Nitsch et al., Ann. Neurol., 48:913-918, 2000.
Nitsch et al., Science, 258:304-307, 1992.
Offermanns et al., Proc. Natl. Acad. Sci. USA, 91:504-508, 1994.
PCT Appln. WO 2005/005394
Porter et al., J. Biol. Chem., 1246(24):7675-7682, 1971.
Postina et al., J. Clin. Invest., 113:1456-1464, 2004.
Price and Sisodia, Ann. Rev. Neurosci., 21:479-505, 1998.
Ramamurthy et al., J. Neurosci. Res., 84:1402-1414, 2006.
Regan et al., Mol. Pharmacol., 46:213-220, 1994.
Remington's Pharmaceutical Sciences, 18^{th} Ed. Mack Printing Company, 1990, pp. 1289-1329, 1990.
Roepstorff, EXS., 88:81-97, 2000.
Schwaner et al., Biochim. Biophys. Acta, 1265:8-14, 1995.
Selkoe, Physiol. Rev., 81:741-766, 2001.
Shenker et al., J. Biol. Chem., 266:9309-9313, 1991.
Shineman et al., J. Neurosci., 28:4785-4794, 2008.
Skovronsky et al., J. Biol. Chem., 276:17036-17043, 2001.
Sonnenburg et al., J. Biol. Chem., 265:8479-8483, 1990.
Stoeckli et al., Nat. Med., 7(4):493-496, 2001.
Takach et al., J. Protein Chem., 16:363, 1997.
Tang et al., J. Neurol. Sci., 240:53-58, 2006.
Villanueva et al., Enzyme Microb. Technol., 29:99, 1999.
Wang et al., Annu. Rev. Microbiol., 54:799-825, 2000.
Wang et al., J. Am. Soc. Mass Spectrom., 10(4):329-338, 1999.
Wittmann et al., Biotechnol. Bioeng., 72:642, 2001.
Wu et al., Anal. Chem., 263(2):129-138, 1998.
Wu et al., Anal. Chem., 69(18):3767-3771, 1997.
Yang et al., J. Agric. Food Chem., 48(9):3990-3996, 2000.
Zagol-Ikapitte et al., J. Neurochem., 94:1140-1145, 2005.
Zaluzec et al., Protein Expr. Purif, 6:109-123, 1995.
Zhong et al., Clin. Chem. ACTA., 313:147, 2001.
Zweigenbaum et al., Anal. Chem., 71(13):2294-300, 1999.
Zweigenbaum et al., J. Pharm. Biomed. Anal., 23(4):723-733, 2000.

1. A method for assessing the risk of development or diagnosing the presence of Alzheimer's Disease (AD) in a subject comprising:
(a) obtaining a sample from said subject that contains soluble APP (sAPP) or that contains C-terminal fragment of APP (CTF); and
(b) assessing sAPPα to sAPPβratio or αCTF to βCTF ratio in said sample,
wherein a decreased sAPPα to sAPPβ ratio or a decreased αCTF to βCTF ratio is indicative of a decreased α-secretase activity with respect to β-secretase activity, which is indicative of risk or presence of AD.

2. The method of claim 1, wherein said sample is cerebrospinal fluid, whole blood, plasma, serum or a biopsy.

3. The method of claim 1, wherein assessing comprises mass spectrometry, radioactive immunoassay (RIA), enzymatic immunoassay (EIA), or enzyme-linked immunosorbent assay (ELISA).

4. The method of claim 1, further comprising isolating sAPP or CTF prior to step (b).

5. The method of claim 3, further comprising digesting sAPP with a protease that generates at least one a distinct peptide from sAPPα or sAPPβ.

6. The method of claim 5, wherein said distinct peptide is MDAEFR.

7. The method of claim 5, wherein said protease further produces at least one common peptide from sAPPα and sAPPβ.

8. The method of claim 7, wherein said common peptide is TEEISEVK (SEQ ID NO:1).

9. The method of claim 5, wherein said protease is trypsin or endoproteinase ArgC.

10-15. (canceled)

16. A method for assessing efficacy of an Alzheimer's Disease (AD) treatment in a subject comprising:
(a) obtaining a first sample from said subject that contains soluble APP (sAPP) or that contains C-terminal fragment of APP (CTF);
(b) obtaining a second sample from said subject that contains sAPP or CTF; and
(c) assessing sAPPα to sAPPβ ratio or αCTF to βCTF ratio in both of said samples,
wherein an increased sAPPα to sAPPβratio or αCTF to βCTF ratio in said second sample as compared to said first sample is indicative of a treatment efficacy.

17. The method of claim 16, wherein said sample is cerebrospinal fluid, whole blood, plasma, serum or a biopsy.

18. The method of claim 16, wherein assessing comprises mass spectrometry, radioactive immunoassay (RIA), enzymatic immunoassay (EIA), or enzyme-linked immunosorbent assay (ELISA).

19. The method of claim 16, further comprising isolating sAPP or CTF prior to step (b).

20. The method of claim 18, further comprising digesting sAPP with a protease that generates at least one a distinct peptide from sAPPα or sAPPβ.

21. The method of claim 20, wherein said distinct peptide is MDAEFR (SEQ ID NO:2).

22. The method of claim 18, wherein said protease further produces at least one common peptide from sAPPα and sAPPβ.

23. The method of claim 22, wherein said common peptide is TEEISEVK.

24. The method of claim 20, wherein said protease is trypsin or endoproteinase ArgC.

25-30. (canceled)

31. A method of screening a candidate substance for activity against Alzheimer's Disease (AD) comprising:
(a) providing a cell that produces soluble APP (sAPP) or C-terminal fragment of APP (CTF);
(b) contacting said cell with said candidate substance;
(c) obtaining sAPP or CTF from said cell; and
(d) assessing sAPPα to sAPPβ ratio or αCTF to βCTF ratio in said sample,
wherein an increase in sAPPα to sAPPβ ratio or αCTF to βCTF ratio is indicative of candidate substance activity against AD.

32-35. (canceled)

36. A method for assessing progression of Alzheimer's Disease (AD) in a subject comprising:
(a) obtaining a first sample from said subject that contains soluble APP (sAPP) or C-terminal fragment of APP (CTF);
(b) obtaining a second sample from said subject that contains sAPP or CTF; and
(c) assessing sAPPα to sAPPβ ratio or αCTF to βCTF ratio in both of said samples,
wherein a decreased sAPPα to sAPPβ ratio or αCTF to βCTF ratio in said second sample as compared to said first sample is indicative of a AD progression.

37. A method for staging Alzheimer's Disease (AD) in a subject comprising:
(a) obtaining a sample from said subject that contains soluble APP (sAPP) or C-terminal fragment of APP (CTF);
(b) assessing sAPPα to sAPPβ ratio or αCTF to βCTF ratio in said sample; and
(c) comparing said sAPPα to sAPPβ ratio or αCTF to βCTF ratio to known ratios for stages of AD.

38-44. (canceled)
